(19) United States
(12) Patent Application Publication
(10) Pub. No.: US 2023/0090830 A1
(43) Pub. Date: Mar. 23, 2023

(54) SWELLABLE ANTIMICROBIAL FIBRE

(71) Applicant: IO-CYTE LIMITED, Leeds West Yorkshire (GB)

(72) Inventor: Christopher Ochayi AGBOH, Leeds West Yorkshire (GB)

(21) Appl. No.: 17/801,900

(22) PCT Filed: Oct. 22, 2020
(86) PCT No.: PCT/GB2020/052662

§ 371 Date: Aug. 24, 2022

(30) Foreign Application Priority Data
Oct. 24, 2019 (GB) 1915425.1

(51) Int. Cl. A61L 15/44; A61L 15/28; A61L 15/60

(52) U.S. Cl. CPC A61L 15/44; A61L 15/28; A61L 15/60; A61L 2300/202; A61L 2300/602

(57) ABSTRACT

A swellable polymer based fibre and method of preparing the same for use as, for example, a wound dressing. The fibres may be formed from an aqueous dope solution containing a primary polymer and povidone-iodine (PVP-I). The aqueous dope solution is spun or extruded into a coagulation bath via a multi-hole spinneret head to form a multifilament fibre.

### FIELD OF INVENTION

The present invention relates to a swellable polymer fibre incorporating an antimicrobial and a method of producing the same and in a particular, although not exclusively, to a spun or extruded swellable polymer fibre incorporating an antimicrobial agent for use as a wound dressing.

### BACKGROUND ART

A variety of different dressings have been developed for treating many different types of wounds from grazes and cuts to more serious and problematic wounds such as burns or ulcers. In particular, these latter types of wound tend to produce significant quantities of exudate which more conventional pads and bandages cannot absorb.

Wound dressings may be formed from gauzes, films, woven or non-woven fabrics and swellable materials including hydrocolloids, alginates, hydrogels, polysaccharides. Such wound dressings may be natural or synthetic and are designed specifically for their biocompatibility.

For major wounds such as burns or ulcers it can be advantageous to prevent infection by introducing an antimicrobial agent to the wound. This may be applied directly before a fresh dressing is applied or more recently the antimicrobial may be incorporated into the dressing which is then placed directly onto the wound. The therapeutic and antimicrobial properties of iodine have been known for centuries with iodine-rich plants being used in the preparation of topical pastes to reduce pain and to help with wound healing. Iodophors are complexes of iodine and a solubilizing agent or carrier. The carrier also functions to control dissociation of the iodine to provide a sustained release when incorporated in a wound dressing and present on a wound in contact with wound exudate. A particular iodophor that has found more recent medical application is povidone-iodine (PVP-I) which is a complex salt of polyvinylpyrrolidone with triiodide ions. The antimicrobial activity of an iodophor, present within a wound dressing, is dependent on the amount of ‘free’ iodine, alternatively-termed ‘available’ iodine that can be released into or onto the wound. This available iodine may be identified quantitatively via iodometry.

WO 2013/140362 A1 discloses a polymeric composite material having antimicrobial and biodegradable properties. The material is used to form medical devices having antiseptic action that is formed from a matrix of alginate and PVP-I. The composite material is used for producing films, micro-capsules and suture threads from which iodine may be released.

EP 0532275 B1 describes a wound dressing having an anhydrous water-soluble gel formed from a polysaccharide or cellulosic polymer together with a humectant. The dressing may also comprise a medicament or additive such as chlorohexidine, a silver compound or an antimicrobial such as PVP-I.

US 2011/0171284 A2 describes a medical dressing for wound healing including a sucrose, PVP-I and a gelling agent sufficient to thicken the composition to control release of sucrose and iodine to the wound.

WO 2013/078998 A1 describes a slow-release ophthalmic composition containing PVP-I to treat acute ophthalmic infections. The composition includes a pharmaceutically acceptable excipient (e.g. water) and PVP-I formed in microspheres with sodium alginate,

However, iodine-based antimicrobial wound dressings are required offering enhanced exudate management and sustained and controlled release of the pharmaceutically active agent.

### SUMMARY OF INVENTION

It is an objective of the present invention to provide a pharmaceutically active fibre, filament or yarn to form a material suitable as a wound dressing exhibiting moisture absorbing characteristics whilst providing a sustained and controlled release of the antimicrobial agent. It is a further objective to provide a fibre and/or a non-woven felt like material having the desired physical and mechanical characteristics for the treatment of a variety of different types of wound from minor cuts and grazes to more serious forms such as burns and ulcers.

The objectives are achieved by providing a fibre, yarn, multi filament and/or material exhibiting enhanced moisture absorbing qualities together with a controlled and sustained release of iodine (as an antimicrobial agent). In particular, the present fibre and material when positioned at a wound are effective to achieve a desired moisture vapour transmission rate from the wound and through the material, a desired physical integrity so as not to degrade when absorbing moisture and exudate, moisture retention so as to provide a hygroscopic humectant whilst also enabling convenient release or decoupling from the wound when required.

According to a first aspect of the present invention there is provided a swellable fibre formed by a method of extrusion of a primary polymer comprising the steps of creating an aqueous dope solution containing a primary polymer and povidone-iodine; spinning or extruding the dope solution into a coagulation bath via a spinneret having a plurality of holes to form an extruded multi filament fibre; and drawing the fibre from the coagulation bath.

The present materials and processes are designed specifically to provide moisture absorbing, antimicrobial structures having a desired moisture vapour transmission rate (MVTR), physical integrity—so as not to degrade when exposed to exudate and in a moisture absorbed swollen configuration in addition to providing controlled and sustained antimicrobial release at the wound

The present materials and methods may utilise a variety of different primary polymers including water-soluble polymers being natural and/or synthetic materials. Such primary polymers are moisture absorbing swellable polymers and include for example any one or a combination of a polysaccharide; a polysaccharide based material; or a hydrocolloid forming material.

Optionally, the primary polymer comprises any one or a combination of an alginate, chitosan, chitin, pectin, carboxymethyl cellulose, -hydroxypropyyl methylcellulose, gellan, psyllium or konjac.

Optionally, the primary polymer is a high guluronic acid (G) or high mannuronic acid (M) acid content alginate, optionally having a molecular weight in the range 32,000 and 400,000 g/mol.

Optionally, a concentration of the povidone-iodine in the fibre is in a range 0.5 to 40 wt %, 0.5 to 30 wt %, 0.5 to 25 wt % or 1.0 to 20 wt % or 0.5 to 20 wt % based on a total weight of the fibre/material.

According to a further aspect of the present invention there is provided a swellable fibre comprising an elongate multi filament body formed from a plurality of intertwined filaments, each filament comprising a primary polymer and povidone-iodine incorporated within the respective filament.

Optionally, the primary polymer comprises a high G-alginate or a high-M-alginate and a povidone-iodine concentration in the range 0.5 to 20 wt % based on a total wt % of the fibre.

Optionally, the primary polymer swellable fibre comprises psyllium.

Preferably, the present fibre and methods utilise additional compounds to further control the release of iodine from the fibre. Such additional components may include secondary water-soluble control compounds such as a water-soluble control compound that comprises a polyether, an alkyl ether, a compound having a C—O—C linkage, a polyol, a glycol or any one or a combination of polyethylene glycol and-propylene glycol. In particular, it is preferred that the water-soluble control compound is polar or at least partially polar. It is hypothesised that the water-soluble control compound may interact via electrostatics and/or structural conformation so as to at least partially inhibit disassociation of the iodine from the PVP complex. Accordingly, the iodine is inhibited from uncontrolled or free release from the material by interaction of the iodine and/or the iodine complex with the water-soluble control compound.

Preferably, the water-soluble control compound comprises propylene glycol and/or polyethylene glycol, wherein a concentration of the water-soluble control compound is such so as to provide the desired sustained release of iodine from the material when exposed to moisture/exudate at the wound. Such a concentration within the final material (suitable for use as a wound dressing) may be in the range 0.5 to 40 wt %, 0.5 to 30 wt %, 0.5 to 25 wt % or 1.0 to 20 wt % based on a total weight of the material.

Optionally, the povidone-iodine is present internally within the multi filament fibre at or towards a core of each filament of the multi filament fibre and at an external surface of the multi filament fibre.

According to a further aspect of the present invention there is provided a method of forming a swellable polymer based fibre comprising: creating an aqueous dope solution containing a primary polymer and povidone-iodine; spinning or extruding the dope solution into a coagulation bath via a spinneret having a plurality of holes to form an extruded multi filament fibre; and drawing the fibre from the coagulation bath.

Optionally, the dope solution may comprise a water-soluble control compound comprising a polyether, an alkyl ether, a compound having a C—O—C linkage, a polyol, a glycol or any one or a combination of polyethylene glycol and propylene glycol.

Optionally, the coagulation bath comprises an aqueous solution containing calcium chloride and any one or a combination of polyethylene glycol, propylene glycol or isopropanol.

Optionally, the coagulation bath further comprises povidone-iodine.

Optionally, the step of drawing the fibre from the coagulation bath comprises passing the fibre into at least one washing bath containing a washing liquid. Optionally, the washing liquid may comprise acetone and/or isopropanol. Optionally, the washing liquid may further comprise any one or a combination of polyethylene glycol, propylene glycol, a finishing agent, water and/or povidone-iodine.

Optionally, the method may comprise passing the extruded fibre into a plurality of washing baths in-series. Optionally, at least some of the washing baths have different washing liquids with different respective constituents.

Preferably, the PVP-I dope, coagulation bath and/or washing bath(s) further comprises a polar organic solvent. The polar organic solvent is advantageous as a substitute to reduce the amount of water in the respective aqueous solution. This has been found to facilitate drying of the processed material to remove excess liquid. Incorporating a polar organic solvent within the solution with which the present material is contacted, is further beneficial to increase the softness of the final material. Such a property is advantageous for use as a wound dressing as will be appreciated. Optionally, the polar organic solvent comprises any one or a combination of an aldehyde, a ketone, an alcohol, an acetal or a compound with a hydroxyl group or a carbonyl group. More preferably, the polar organic solvent may comprise acetone and/or isopropanol.

Importantly, the present PVP-I fibre, once manufactured, comprises a desired moisture content. This avoids the resultant fibres agglomerating which is particularly important for the multi filament processing. Additionally, it is important to provide a resultant material that comprises a generally uniform structure devoid of cracks or undesired large internal cavities or voids otherwise associated with poorly spun fibres A PVP-I fibre having a desired moisture content would facilitate crimping, opening, carding and conversion into wound dressing with the desired physical and mechanical characteristics such as the desired moisture vapour transmission rate, exudate absorption etc. Optionally, the present PVP-I fibre comprises a moisture or liquid content in the range 5 to 60%, 10 to 60%, 15 to 55%, 20 to 50%, 25 to 50%, 30 to 50%, 30 to 45% or 35 to 40% moisture. The moisture content may be determined by any suitable method. For example, the moisture content may be determined by subtracting the dry weight of the fibre from the appropriately moistened fibre and then dividing this difference (moisture content) by the total weight of fully moistened fibre. The values of moisture content reported herein therefore are relative moisture wt % ranges of the amount of liquid within the moistened fibre on the processing line.

The present PVP-I fibre may comprise a moistening liquid being any one or a combination of water, a water-based solution, an organic liquid, an organic solution, acetone, isopropanol.

Optionally, the present fibre and/or material may comprise at least one additional or further antimicrobial agent. The further antimicrobial agent may comprise silver, a silver ion or a silver containing compound. Optionally, the further antimicrobial agent comprises a metal species being one or a combination of the set of Zn, Cu, Ti, Pt, Pd, Bi, Sn, Sb.

Preferably, the method further comprises cutting the PVP-I fibres and compressing, squeezing or pressing the moistened fibre to expel excess moisture prior to opening, carding and before allowing the fibre to dry.

Optionally, fibres produced may be processed in dry form and more preferably are processed in damp form with the fibre retaining 30-45% moisture.

According to a further aspect of the present invention there is provided a non-woven felt-like material comprising a swellable fibre as claimed herein. Optionally, the material is a wound dressing material.

According to a further aspect of the present invention there is provided a material comprising a swellable fibre as described and claimed herein, the material being any one of: a wound dressing material; a nasal packing material; a dental packing material; a suture; a seton. Optionally the present material when used as a wound dressing or packing material is non-woven. Optionally the present material when used as a suture or a seton is fibrous.

### BRIEF DESCRIPTION OF DRAWINGS

A specific implementation of the present subject matter will now be described, by way of example only, and with reference to the accompanying drawings in which:

FIG. 1 is a cross-sectional view of apparatus used in wet spinning a swellable polymer fibre according to a specific implementation of the present invention;

FIG. 2 is a graph of viscosity of PVP-I solutions against spindle speed;

FIG. 3 is a graph of viscosity against shear or spindle speed to investigate the effect of addition of PVP-I on alginate-based dope together with viscosity, aging and type of alginate used;

FIG. 4 is a graph of viscosity versus shear or spindle speed of Acros-PVP-I aqueous solutions at different concentrations;

FIG. 5A is a graph of viscosity against shear or spindle speed profile of a PVP-I complex;

FIG. 5B is a graph of viscosity versus shear or spindle speed profile of PVP-I aqueous solutions;

FIG. 6 is a graph of viscosity and shear spindle speed profile of high M-alginate-PVP-I dope solutions in examples 16 and 17;

FIG. 7 is a graph of viscosity versus shear spindle speed profile with addition of psyllium in alginate PVP-I dope solutions.

### DETAILED DESCRIPTION

The present antimicrobial swellable fibre is suitable for the manufacture of a non-woven felt-like material that, in turn, may be utilised as a wound dressing for the treatment of a variety of different types of wound from cuts and grazes to more serious burns, ulcers and the like where exudate management is critical. The present fibre is conveniently formed from a multifilament via an extrusion or spinning process in which the fibre is spun or extruded from an aqueous dope solution into a coagulation solution. The present materials and processes provide spinning, extruding conditions and processing parameters to produce highly swellable fibres incorporating ‘available’ iodine within the fibre core and at the fibre surface. The iodine is in a form of an iodine complex and included at a concentration level sufficient to provide antimicrobial activity. Such concentration levels may be of the order of greater than 1 wt % ‘free’ iodine within a total weight of fibre. The present materials and processes are specifically designed to provide a material having a desired moisture vapour transmission rate (MVTR), physical integrity—so as not to degrade when exposed to exudate and in a moisture absorbed swollen configuration, in addition to providing controlled and sustained antimicrobial release at the wound.

The present materials and methods may utilise a variety of different primary polymers including natural or synthetic materials. Such primary polymers are moisture absorbing swellable polymers and include for example polysaccharides or polysaccharide based materials, hydrocolloids, biopolymers. A preferred form of primary polymer is a polysaccharide alginate.

The present fibre and methods may utilise additional compounds to further control the release of iodine from the fibre. Such additional components may include secondary water-soluble control compounds such as polyethers, alkyl ethers, a glycol such as propylene glycol or a compound having a C—O—C linkage such as or polyethylene glycol (PEG).

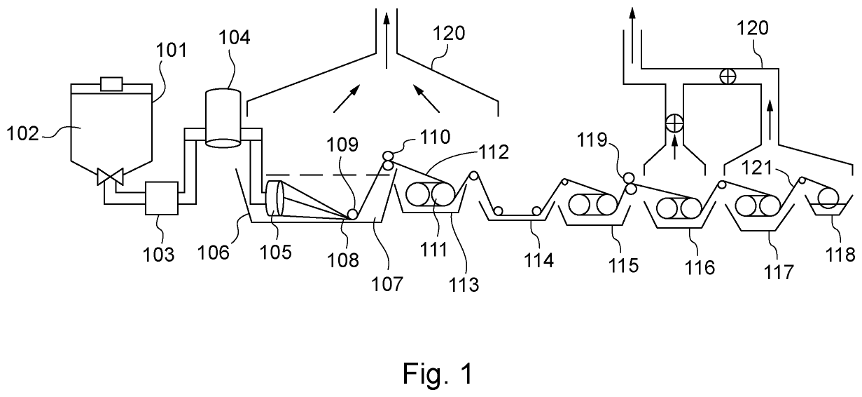

Referring to FIG. 1, swellable polymer fibres are produced by first dissolving component materials (polymer species and other additives, as detailed in the following examples) in water to form a dope solution 102. The dope solution is contained within a vessel 101 under an inert atmosphere. Dope solution 102 is then passed directly through a pump 103 which increases the pressure of the system. Solution 102 is then filtered via a filter 104, before entering a multi-hole/multi-aperture spinneret head 105. Solution 102 is then extruded into the spinneret head 105 and then immersed in a coagulant 107 contained within a coagulation bath 106 to form tow/coagulated filaments 108. These filaments are then hauled-off bath 106 over filament guides 109 before passing between mangling rollers 110 that act to squeeze the fibres to expel liquid/moisture from the coagulation and dope solutions.

The partially dried fibres 112 are then passed into a first wash bath 113 via advancing rollers 111. Subsequently, the fibres 112 are passed into successive baths 114, 115, 116 and 117. Such baths may contain the same or different washing solutions including combinations of water, alcohols, organic solvents and further finishing compounds. At a stage between baths 115 and 116, the fibres 112 are squeezed again by mangling rollers 119 for further moisture removal. The washed fibres 121 are then passed into a final bath 118 and/or winding unit for collection and onward dispatch.

The spinning line further comprises extraction systems 120 to remove moisture and solution vapour from coagulant bath 106 and washing baths 113 to 118.

As will be appreciated, the example spinning line for the manufacture of swellable fibres in accordance with the present disclosure may comprise additions or variations to the components described and illustrated referring to Example 1. Additionally, spinneret head 105 comprises a plate having a plurality of holes, apertures or capillaries through which the dope solution is passed to form the coagulated tow. A multi-hole spinneret accordingly provides resulting multifilaments being a collection of monofilaments associated, spun and/or entrained together to form a collective fibre assembly. As will be appreciated, such a multifilament fibre may then be processed by subsequent downstream operations to create a non-woven fibrous material suitable for use as a wound dressing or the like.

The following examples are based on an alginate fibre incorporating polyvinyl pyrrolidone (PVP-I). However, as indicated, the present material and method may equally comprise other swellable polymers and variations of spinning or extrusion processes.

### Example 1

In this example, the preparation and effect of solid content on the viscosity of PVP solution alone or with sodium alginate was investigated in addition to the effects on the spinnability and fibre properties with the addition of PVP-I to alginate solutions.

Aqueous solutions of 2, 10 and 20% w/w PVP solutions were easily prepared by mixing 5 g, 25 g and 50 g of PVP powder (lot SLBV2087, m.pt 150-180° C. supplied by Sigma-Aldrich) into 245 g, 225 g and 200 g of water respectively. After deaeration, the viscosity of each solution at 25° C. was determined using a Brookfield digital viscometer (RVTD) and RV spindle size 04. The pH of the solutions appeared to change from 6 for the 2% w/w solution to about 4 for the 20% w/w solution. The reasons were not clear but not thought to be significant in the fibre extrusion when added to alginate dopes.

TABLE 1
Effect of solid content and shear rate
on viscosities of PVP solutions

| | Solution concentration, % w/w | | |
|---|---|---|---|
| | 2 | 10 | 20 |
| Shear speed, rpm | Viscosity, cp | | |
| 20 | 5.0 | 10 | 22.5 |
| 50 | 8.0 | 16 | 31.0 |
| 100 | 12.0 | 22.5 | 40.0 |

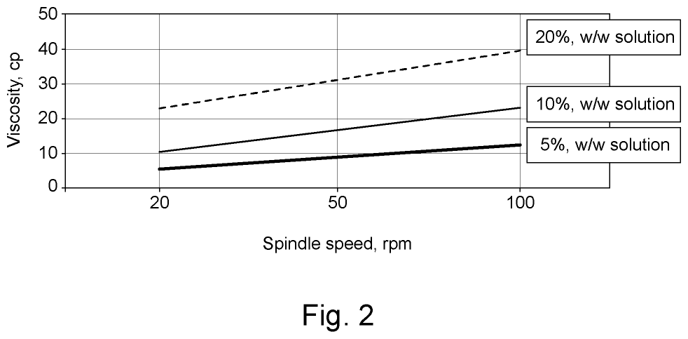

Referring to FIG. 2, which shows the effect of solid content and shear speed on viscosity of PVP solutions, the solutions appeared to increase in viscosity with shear speed (indicating a shear thickening or dilatant properties) which was peculiar as this type of behaviour are mostly associated with mixtures and suspensions and hardly with pure well dissolved polymer solutions. The behaviour has been associated with the very low nature of viscosities obtained and perhaps the limits of the spindle size used. The important aspect of the results was to indicate that addition of the polymer to alginate solutions would not alter the viscosities significantly as demonstrated in the next examples.

### Example 2

### Simple Rheology of Alginate-PVP Dopes

This example describes the preparation of High G or High M based calcium alginate/PVP dopes and their viscosity assessment. For the High G alginate, the 15% w/w dope concentration and weight 400 g contained 5% w/w alginate and 10% w/w PVP. The dope was prepared by first manually mixing 20 g of alginate powder with 40 g of PVP powder and then gradually adding the mixture to 340 g of water that was being stirred vigorously with a high shear mixer. The mixture was stirred continuously for 1 h to ensure a homogeneous dope. The viscosity was then assessed as in Example 1 before and after standing overnight.

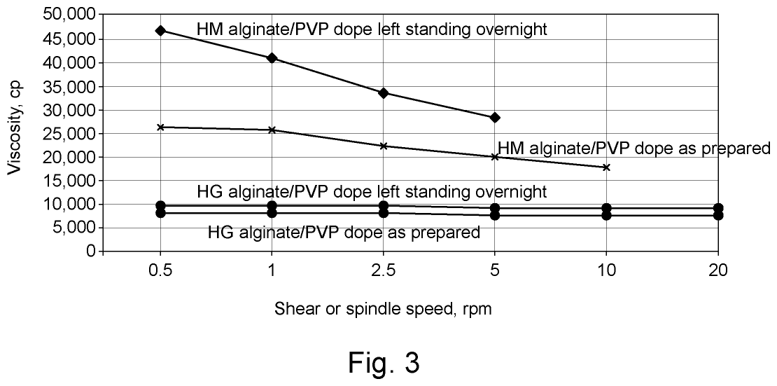

The procedure was repeated for High M alginate to obtain the results displayed in Table 2 and FIG. 3 which is a graph showing the effect of addition of PVP on alginate dope viscosity, aging and type of alginate used.

TABLE 2
Effect of addition of PVP on alginate dope
viscosity, ageing and type of alginate used

| | Dope concentration, 15% w/w | | | |
|---|---|---|---|---|
| | Alg./PVP concentrations, % w/w in the dope | | | |
| | HG-180603-PVP | | HM-180603-PVP | |
| | Alginate | PVP | Alginate | PVP |
| | 5% w/w | 10% w/w | 5% w/w | 10% w/w |
| | Dope viscosity, cp | | Dope viscosity, cp | |
| Spindle | As soon as | Deaerated | As soon as | Deaerated |
| or Shear | prepared | after | prepared | after |
| speed, | deaeration | standing | deaeration | standing |
| rpm | (no deaearation) | overnight | (no deaearation) | overnight |
| 0.5 | 8,000 | 9,600 | 26,400 | 46,800 |
| 1.0 | 8,000 | 9,400 | 25,600 | 41,000 |
| 2.5 | 8,000 | 9,400 | 22,320 | 33,600 |
| 5.0 | 7,880 | 9,280 | 19,840 | 28,480) |
| 10.0 | 7,700 | 9,180 | 17,480 | — |
| 20.0 | 7,530 | 8,950 | — | — |

The results indicate the addition of PVP to High G alginate solution appeared to have very little effect on the viscosity and behaviour of the solution even on ageing suggesting that a substantial percentage of PVP could be used if necessary, to potentially change fibre properties. On the other hand, addition of 10% w/w PVP to a 5% w/w High M alginate solution produced a very high viscous dope with tendency to gel on standing. This demonstrates great potential to produce highly absorbent fibres from High M/PVP fibre. However, fibre production may be affected by dope gelation and uncontrollable swelling in the bath. A very low percentage of PVP in the dope may be advantageous.

### Examples 3 to 7

High G Alginate-PVP Fibres

These examples describe the preparation of High G based calcium alginate/PVP dopes and production of fibres from the dopes.

The dopes were prepared using any of the following general approaches:
(1) PVP powder was dissolved in water before gradually adding the Na— alginate powder to the dissolved PVP solution and mixing vigorously to obtain a homogeneous dope;
(2) the PVP was manually mixed with the Na-alginate powder. The mixed powders were then added gradually to stirred water and followed by vigorous mixing to obtain a homogenous dope;
(3) alternatively, the Na-alginate powder may be stirred into water and whilst dissolving, the PVP powder may be added gradually and mixed well to obtain a homogeneous dope.

A high shear mixer at 3000-4000 rpm was used (forward and/or reverse direction) for 15 minutes before and after addition of powder. The mixer speed was then reduced to 2,000 rpm and left for 30 to 45 minutes depending on dope concentration and viscosity. Once homogeneously mixed, the dope was either vacuum de-aerated or allowed to stand until fully deaerated.

TABLE 3
Preparation of High G alginate/PVP dope solutions for extrusion

| | | | | | Example 5 | | | | | |
|---|---|---|---|---|---|---|---|---|---|---|
| | | | | | HG-180626-PVP | | | | Example 7 | |
| | Example 3 | | Example 4 | | HG-180627-PVP | | Example 6 | | HG-180704-PVP | |
| Dope Preparation | HG-180615- | | HG-180622- | | (Example 5 | | HG-180702- | | (Example 4 | |
| Parameters | PVP | | PVP | | repeated) | | PVP | | repeated) | |
| Dope concentration, % w/w | 8 | | 5 | | 6 | | 5 | | 5 | |
| | Alg. | PVP | Alg. | PVP | Alg. | PVP | Alg. | PVP | Alg. | PVP |
| Alg./PVP concentration, % | 5 | 3 | 4.25 | 0.75 | 5.4 | 0.6 | 4.5 | 0.5 | 4.25 | 0.75 |
| w/w in the dope | | | | | | | | | | |
| Expected Alg./PVP | 62.5 | 37.5 | 85 | 15 | 90 | 10 | 90 | 10 | 85 | 15 |
| concentrations in the fibre | | | | | | | | | | |

| | Dope preparation | | | | |
|---|---|---|---|---|---|
| Dope wt., g | 1,500 | 1,500 | 2,000 | 2,000 | 2,000 |
| Alginate wt., g | 75 | 63.75 | 108 | 90 | 85 |
| PVP wt., g | 45 | 11.25 | 12 | 10 | 15 |
| Water wt., | 1,380 | 1,425 | 1,880 | 1,900 | 1,900 |

For example, in Example 3, a spinning dope of solid content 8% (w/w) and weight 1,500 g was prepared by mixing together 75 g (5% w/w) sodium alginate powder with 45 g (3% w/w) PVP powder, and then gradually adding the mixed powders to 1380 g of water. The dope was then allowed to stand for three days to deaerate. After viscosity assessment, the dope was passed under pressure through a 25μ cartridge filter and extruded. Similar procedures were repeated for examples 3-7.

Once filtered, each dope was spun through a 90μ/2,000-hole spinneret into a calcium chloride dihydrate coagulation bath and hauled-off whilst simultaneously being washed in water placed under the haul-off rollers. From the haul-off bath, the fibres were stretched through an orientation bath containing acetone/water (56/24, v/v), then washed through successive acetone/water baths at varying compositions before finally washing in pure acetone bath and winding onto a drum roller. The fibres were cut off from the drum roller, hand crimped and then dried at ambient. Table 4 below shows the conditions used to spin each batch and type of fibres obtained.

TABLE 4
Fibre extrusion conditions for examples 4-7

| | Dope viscosity, | | Amount of | | Draw speed, m/min | | | |
|---|---|---|---|---|---|---|---|---|
| | cp | | CaCl₂•2H₂O | Haul off | (washing bath composition: Acetone/water, | | | |
| Example/ | (at 5 rpm | Extrusion rate, | in the coag. | speed, | v/v) | | | |
| Batch no. | shear speed) | m/min | Bath, % | m/min | D1 | D2 | D3 | Winder |
| Example 3/ | 3,760 | 3.9 | 1.5 | 2.9 | 4.3 | 4.4 | 4.4 | 4.5 |
| HG-180615- | | | | (0/100) | 50/50 | 80/20 | 100/0 | 100/0 |
| PVP | Moderate jet stretch, JSR ratio (0.74) and high draw ratio, DR (1.48): | | | | | | | |
| | With this pump speed, the extrudates appeared too relaxed in the bath and very highly swollen but separated and | | | | | | | |
| | strong. Fibres were difficult to dry, harsh, tough and stuck together even in the pure final acetone bath. Not much | | | | | | | |
| | difference was observed at lower extrusion rates. Fibres obtained were highly absorbent for High G alginate, but | | | | | | | |
| | fluid retentions were rather low indicating that the fibres were swollen but not gelled in the fluids used: | | | | | | | |
| | Absorbency in solution A: (1-30 mins) 21 g/g and fluid retention 45 g %. No difference in immersion time. | | | | | | | |
| | Absorbency in saline: 18.5 g/g retention 44% (1 min) and after 30 mins, 22 g/g and retention 55%. Appeared to | | | | | | | |
| | show the expected results that both the absorbency and retention increased with time of immersion in the fluid | | | | | | | |
| Example 4/ | — | 2.8 | 1.8 | 2.95 | 3.66 | 3.80 | 3.81 | 3.80 |
| HG-180622- | | | | 51/49 | 77/23 | 96/4 | 98/2 | — |
| PVP | Moderate jet stretch, JSRratio (1.05) and high draw ratio, DR(1.48): | | | | | | | |
| | Orientation bath: acetone/water 56/44, v/v (circulated) | | | | | | | |
| | Fibre still swollen especially on the haul-off rollers with 100% water as washing bath even with reduced amount | | | | | | | |
| | of PVP in the dope. Low draw ratio caused fibres to stick to the rollers and made extrusion difficult with frequent | | | | | | | |
| | breakages. On the other hand, high stretching and rapid washing in acetone/water baths appeared to cement the | | | | | | | |
| | fibres more together preventing good separation. Replacing the haul-off washing liquid with acetone/water and | | | | | | | |
| | with moderate fibre stretching improved the spinning, openness and reduced brittleness but fibres still difficult to | | | | | | | |
| | dry. Nevertheless, these spinning conditions appeared to have produced better fibres than before. Generally | | | | | | | |
| | though, it appeared that: | | | | | | | |
| | i) PVP appeared to cause the freshly spun fibre to retain a lot of fluid in the fibre ii) washing in water swelled the | | | | | | | |
| | fibre even more since PVP is soluble in water iii) washing in acetone hardened the PVP quickly in the fibres | | | | | | | |
| | cementing the filaments together and forming very hard and brittle tows iv) a final wash in alcohol, e.g. methanol | | | | | | | |
| | appeared to reduce fibre harshness but no improvement in absorbency noticed. | | | | | | | |
| | Undrawn fibre: | | | | | | | |
| | Absorbency in solution A: 22.2 g/g retention 39% (1 min) and after 30 mins, 21.4 g/g and retention 49%. | | | | | | | |
| | Absorbency in saline: 18.9 g/g retention 44% (1 min) and after 30 mins, 22 g/g and retention 55%. Appeared to | | | | | | | |
| | show the expected results that both the absorbency and retention increased with time in the two fluids. Fibres | | | | | | | |
| | were fully opened. | | | | | | | |
| | Drawn fibres | | | | | | | |
| | Absorbency in solution A: 17.7 g/g retention 31% (1 min) and after 30 mins, 22.4 g/g and retention 38%. | | | | | | | |
| | Absorbency in saline: 19.3 g/g retention 37% (1 min) and after 30 mins, 23.6 g/g and retention 52%. | | | | | | | |
| | The fibres were less opened and harsher | | | | | | | |
| Example 5/ | 4,360 | 2.4 | 1.5 | Haul-off | D1 | D2 | D3 | Winder |
| HG-180626- | | | | 2.95 | 3.66 | 3.9 | 3.9 | 3.87 |
| PVP | | | | 56/24 | 90/10 | 99/1 | 99/1 | — |
| | High jet stretch, JSR ratio (1.2) and moderate draw ratio, DR (1.24) | | | | | | | |
| | Orientation bath: acetone/water 75/25, v/v (circulated) | | | | | | | |
| | Fibres still appeared harsh and brittle in pure acetone baths but after crimping and drying relaxed, they could | | | | | | | |
| | be easily opened. The fibres appeared soft and smooth but too much acetone used in the process showed it | | | | | | | |
| | could not be sustained. Attempts to dry in alcohol, e.g. methanol proved possible but fibre harshness and low | | | | | | | |
| | fluid retention were not really significantly reduced. | | | | | | | |
| | Absorbency in solution A: 21.57 g/g retention 30% (1 min) and after 30 mins, 23.62 g/g and retention 38% | | | | | | | |
| | Absorbency in saline: 20.42 g/g retention 40% (1 min) and after 30 mins, 20.66 g/g and retention 43%. The | | | | | | | |
| | fibre was highly absorbent, integral but did not gel and had low fluid retention | | | | | | | |
| Example 5/ | 4,360 | 2.26 | 1.8 | 3.0 | 3.04 | 3.14 | 3.14 | 3.09 |
| HG-180627- | | | | Warm H2O | 60/40 | 74/26 | 84/16 | — |
| | | | | Start: 50° C. | | | | |
| | | | | End: 5° C. | | | | |
| PVP/1 | High jet stretch ratio (1.33) and low draw ratio (1.01). | | | | | | | |
| | Orientation bath: acetone/water 77-71/23-29, v/v (beginning-end, circulated) | | | | | | | |
| | Fibres separated but still harsh and difficult to dry. Fibre strength was also low. | | | | | | | |
| | Absorbency slightly decreased (Soln. A 11-16 g/g; saline 12-18 g/g), although retention | | | | | | | |
| | for some reasons not clear has increased (soln. A 50-73%; saline 53-67.5%). | | | | | | | |
| Example 5/ | 4,360 | 2.26 | 1.8 | 1.71 | 1.71 | 1.77 | 1.94 | 1.91 |
| HG-0627- | | | | Warm H₂O | 60/40 | 74/26 | 84/16 | — |
| PVP/2 | | | | Start: 50° C. | | | | |
| | | | | End: 35° C. | | | | |
| | Low jet stretch ratio (0.76) and low draw ratio (1.0). Fibre relaxed in coag. bath | | | | | | | |
| | Orientation bath: acetone/water 77-71/23-29, v/v (beginning-end, circulated) | | | | | | | |
| | Extra threading of tow over guide rollers in the coagulation bath before the haul off rollers take-off | | | | | | | |
| | Tow cut off from wider roller, excess liquid squeezed out and left under extraction system rather | | | | | | | |
| | than leaving in at room temperature. | | | | | | | |
| | Tow opened and separated well on drying but as expected, it had low strength. | | | | | | | |
| | Absorbency (Soln. A: 1 min 14.2 g/g, 30 min 20.2 g/g; saline: 1 min 12.8 g/g, 30 min 22 g/g), and | | | | | | | |
| | retention still low at 49% in soln A and 53% in saline). | | | | | | | |
| Example 6/ | — | 2.26 | 1.8 | 1.71 | 2.0 | 2.12 | 2.12 | 2.2 |
| HG-180702- | | | | Warm H2O | Start: 71/29 | 80/20 | 81/19 | 98/2 |
| PVP | | | | Start: 56° C. | End: 59/41 | 66/34 | 76/24 | 97/3 |
| | | | | End: 35° C. | | | | |
| | Low jet stretch ratio (0.76) and low draw ratio (1.16). Fibre relaxed in coag. bath | | | | | | | |
| | Orientation bath: acetone/water 77-50/23-50, v/v (beginning-end, circulated) | | | | | | | |
| | Fibre arrangement and passage through the coag. Bath the same as for HG-180627-PVP/2 Fibres | | | | | | | |
| | produced were fully opened and soft to handle. The small increase (15%) in the draw ratio | | | | | | | |
| | improved the fibre strength. So far, these conditions appeared to have produced the best fibres. | | | | | | | |
| | But fibre absorbency was rather very low, 11 g/g in soln. A and 15 g/g in saline. Retention was 70.5% | | | | | | | |
| | in soln A and somehow remained low (56%) in saline | | | | | | | |
| Example 7/ | — | 2.7 | 1.8 | 1.70 | 2.06 | 2.15 | 2.19 | 2.14 |
| HG-180704- | | | | Warm H₂O | Start: 59/41 | 66/3465/35 | 76/2476/24 | 98/2 |
| PVP (dope | | | | Start: 60° C. | End: 56/44 | | | 98/2 |
| composition | | | | End: 31° C. | | | | |

| the same as in | Low jet stretch ratio (0.63) and low draw ratio (1.21). Fibre relaxed in coag. bath | | | | |
|---|---|---|---|---|---|
| example 4) | Orientation bath: acetone/water 56/44, v/v (beginning-end, circulated) | | | | |
| | | Solution A | | Saline | |
| | | 1 min | 30 min | 1 min | 30 min |
| | Absorbency | 9.1 | 10.3 | 10.0 | 17.9 |
| | Retention | 61.5 | 80.0 | 65.0 | 65.0 |

Fibres were much more opened and softer but absorbency so low and inconsistent. Reasons unclear.

### Examples 8 and 9

### High M Alginate-PVP Fibres

These examples describe the preparation of High M based calcium alginate/PVP dope and production of fibres from the dope. With reference to Table 5 for the amounts of materials used, the spinning dope of weight 2,000 g was prepared by first, manually mixing together PVP powder and High M sodium alginate powder (High M alginate-Manucol DH supplied by FMC Biopolymers, UK, Ltd) with a spatula. The mixed powders were then gradually added to water stirred continuously with a high shear mixer. Once fully added, the mixture was vigorously mixed to obtain a homogenous dope. The dope was then vacuum de-aerated, filtered under pressure through a 25μ cartridge filter and extruded at 2.7 m/min through a 90μ/2,000-hole spinneret into a 1.6% (w/w) calcium chloride dihydrate coagulation bath. The as-spun fibres were hauled-off at 1.7 m/min from the bath, stretched through acetone/water (56/44, v/v) orientation bath, then washed in series of acetone/water baths (Table 6) before finally washing in pure acetone bath and winding onto a drum roller. The fibres were cut off from the drum roller, hand crimped and then dried under extraction before testing for absorbency.

TABLE 5
Dope composition and solid contents for examples 8 and 9

| | (Example 8)/ | | (Example 9)/ | |
|---|---|---|---|---|
| Dope preparation parameters | HM-180621-PVP | | HM-180704-PVP | |
| Dope concentration, % w/w | 15 | | 5 | |
| Dope composition | Alg. | PVP | Alg. | PVP |
| Alg./PVP concentrations, | 5 | 10 | 4.25 | 0.75 |
| % w/w in the dope | | | | |
| Expected Alg./PVP concentrations | 33.3 | 66.7 | 85 | 15 |
| in the fibre | | | | |

| | | |
|---|---|---|
| Dope wt., g | 2,000 | 2,000 |
| High M Alginate wt., g | 100 | 85 |
| Polyvinylpyrrolidone (PVP), wt., g | 200 | 15 |
| Water, wt., g | 1,700 | 1,900 |

TABLE 6
Spinning conditions for examples 8 and 9

| | | | | Draw speed, m/min | | | |
|---|---|---|---|---|---|---|---|
| | | | | (washing bath composition: | | | |
| Example/ | Amount of CaCl2•2H2O | Extrusion rate, | Haul off speed, | Acetone/water, v/v) | | | |
| Batch no. | in the coag. Bath, % | m/min | m/min | D1 | D2 | D3 | Winder |
| Example 8/ | 1.8 | 2.82 | 2.95 | 4.3 | 4.4 | 4.4 | 4.53 |
| HM-180621- | | | water | 50/50 | 77/23 | 97/3 | 100/0 |
| PVP | High jet stretch ratio (1.05) and high draw ratio (1.46). | | | | | | |
| | The tow was highly swollen and difficult to wash even in the acetone baths, still not | | | | | | |
| | properly separated after coming out of pure acetone baths and therefore harsh to handle. | | | | | | |
| | Increased CaCl₂•2H₂O concn in the coag. bath did not appear to have any effect | | | | | | |
| | | Solution A | | Saline | | | |
| | | 1 min | 30 min | 1 min | | 30 min | |
| | Absorbency | 16.5 | 20.3 | 17.1 | | 25.5 | |
| | Retention | 53.0 | 49.5 | 59.0 | | 52.5 | |
| | Fibres produced had good absorbency although for high M alginate fibre higher values were | | | | | | |
| | expected. They had poor retention, did not appear to gel and were therefore integral. | | | | | | |
| Example 9/ | 1.6 | 2.7 | 1.7 | 2.06 | 2.15 | 2.19 | 2.1 |
| HM-180704- | | | water | 65/35 | 76/24 | 77/23 | 98/2 |

| PVP | Low jet stretch ratio (0.63) and Low draw ratio (1.21). | | | | |
|---|---|---|---|---|---|
| | Orientation bath: acetone/water 56/44, v/v circulated) | | | | |
| | Fibre spun at the beginning of extrusion opened and separated well but towards the | | | | |
| | end of extrusion, they became harsher and more difficult to open indicating the | | | | |
| | need to replenish the baths continuously during extrusion to maintain consistency | | | | |
| | | Solution A | | Saline | |
| | | 1 min | 30 min | 1 min | 30 min |
| | Absorbency | 17.4 | 20.3 | 21.9 | 29.4 |
| | Retention | 82.0 | 90 | 89.5 | 89.0 |

Fibres were absorbent with high fluid retention as they formed gel in the fluids.
The gels were almost dispersible.

### Conclusions for Spinning Alginate/PVP Fibres

The following main conclusions were reached:
Dopes containing very high amount of PVP (up to 20% or more in the dope) can be prepared and spun but dope gelation may occur at very high dope concentrations (>15%, see Table 2, FIG. 3) and extrusion would become more difficult with fibre swelling and sticking in the baths as the amount PVP increased in the dope and in the fibre.
Fibres produced tended always to have harsh handle, brittle and stuck together due to high water retention of PVP in the fibres especially at about 10% PVP in the dope.
Consequently, fibre separation, softness, absorbency and retention appeared to hardly change with spinning conditions.
However, best results were obtained with low jet stretch ratio (0.5-0.7) and low draw ratio (1.0-1.4) with acetone/water washing baths containing between 60-70% acetone in the baths till the last bath where about 98% could be used.
Under these spinning conditions, high absorbency (20 g/g) and retention (90%) in solution A after 30 minutes of immersion were obtained with even higher absorbency (29 g/g) in saline for High M/alginate fibres.

### Examples 10 and 11

### Simple Rheology of Aqueous Solutions of PVPI Complex

These examples describe the attempts to prepare fibres from povidone-iodine (PVPI) complex polymer using samples supplied by BASF and Acros. Initially, it was decided to investigate if the complex (PVP-I) could be spun within a reasonable dope concentration. To do this, various concentrations of PVP-I solutions in water were prepared and examined. The solutions were prepared as described in example 1 except that Povidone-iodine (PVP-I) powders supplied by either BASF or Acros were used. The simple rheology of the solutions prepared and their spinnability prospects obtained are outlined below in Table 7.

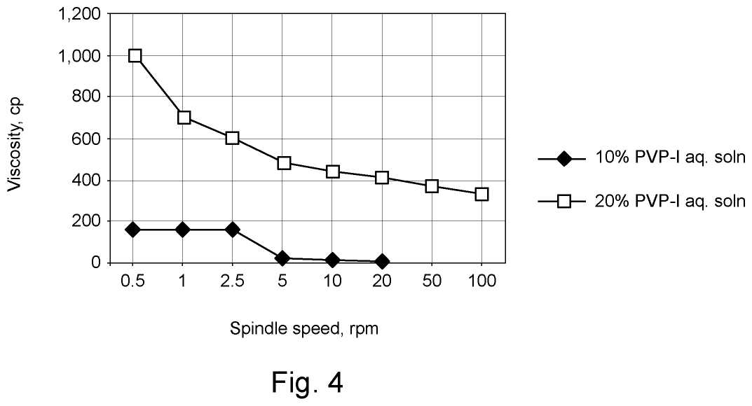

As expected, the viscosity of PVP-I in aqueous solution increased with increasing concentration of PVP-I in solution. However, the viscosities of the solutions prepared were too low even at 20% PVP-I to be spun. The solutions failed to display any spinnable characteristics as the flows were all in droplets. As shown in FIG. 4 which is a graph of the viscosity-shear speed profile of Acrōs PVP-I aqueous solutions, the shear thinning property observed for the 20% solution (Table 7) showed a behaviour typical of solution extrudable viscoelastic polymers, indicating a possible extrusion viscosity at a much higher solution concentration and PVP molecular weights. Indeed, there are several references in the internet that describe the melt spinning and electrospinning of PVP polymer. For PVP-I powders used this study, it was concluded very quickly that the complex solutions alone could not be spun within the concentration range examined.

TABLE 7
Simple rheology and spinning prospects of aqueous povidone-iodine complex solutions.

| | | Composi- | | Spindle speed, rpm/Viscosity, mPa · s or cP | | | | | | | |
|---|---|---|---|---|---|---|---|---|---|---|---|
| | | tion % | | (Spindle size 02; temperature 17° C.) | | | | | | | |
| Example | Batch | PVP-I | water | 0.5 | 1.0 | 2.5 | 5 | 10 | 20 | 50 | 100 |
| 10 | PVP-I 30/06 | 2 | 98 | | | | | | 5 | 7 | 10.5 |
| | (BASF)/2 | | | | | | | | | | |
| | (spindle size | | | | | | | | | | |
| | 03) | | | | | | | | | | |
| | PVP-I 30/06 | 10 | 90 | | | | | | 10 | 14 | 21 |
| | (BASF) | | | | | | | | | | |
| | (spindle size | | | | | | | | | | |
| | 03) | | | | | | | | | | |
| 11 | PVP-I/180124/ | 2 | 98 | — | — | — | — | — | 5 | 8 | 12 |
| | Acrōs/2 | | | Very thin solution with a lot of bubbles which broke easily on standing. | | | | | | | |
| | (spindle size | | | PVP-I/Alg. dopes can be easily prepared with this amount of PVP-I | | | | | | | |
| | 03) | | | | | | | | | | |
| | PVP-I/180124/ | 10 | 90 | 160 | 160 | 160 | 24 | 16 | 16 | 20 | 26 |
| | Acrōs/10 | | | Low viscosity still contained a lot of bubbles and behaviour appears a bit erratic at | | | | | | | |
| | (spindle size | | | high shear rates. But possible to add 10% PVP-I to alg. to prepare a spinnable dope. | | | | | | | |
| | 03) | | | | | | | | | | |
| | PVP-I/180124/ | 20 | 80 | 1,000 | 700 | 600 | 480 | 440 | 415 | 372 | 336 |
| | Acrōs/20 | | | Dope still unspinnable, viscosity still too low | | | | | | | |
| | (Spindle size | | | | | | | | | | |

04)

Note:
PVP-I obtained from Acrōs and claimed to contain 100% poly(vinylpyrrolidone)-iodine complex. Contains no formic acid, solution pH acidic.

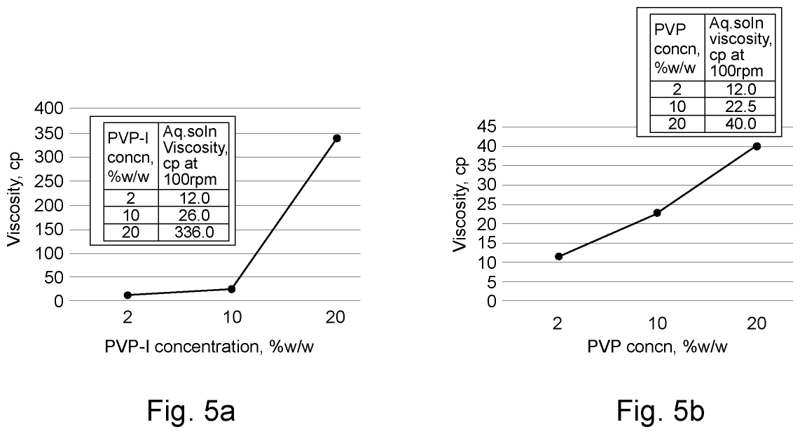

The complex required incorporation of a fibre forming polymer such as alginate for extrusion. FIGS. 5a and 5b are graphs showing the effect of PVP-I concentration on viscosity in aqueous solution. These appear to indicate, complexing iodine with PVP has somehow modified the rheological behaviour of PVP as the viscosity of the complex in aqueous solution appeared to have increased exponentially above 10% concentration. This was significant as it marked the range of spinnable solutions (<10%) without encountering a lot of problems such as excessive swelling, fibre sticking together and drying difficulties during spinning.

### Examples 12 and 13

High G Alginate-PVPI fibres

These examples describe the initial attempts to prepare High G alginate-povidone iodine (PVPI) complex fibres. With reference to Table 8 for the amounts of materials used, the spinning dope of weight 2,000 g or 2,500 g was prepared by first, manually mixing together PVP-I powder (30/06 supplied by BASF) and High G sodium alginate powder (High G alginate-Protanal LF10/60 FT supplied by FMC Biopolymers, UK, Ltd) with a spatula. The mixed powders were then gradually added to water that was being stirred continuously with a high shear mixer. Once fully added, the mixture was vigorously mixed for 1 h to 1.5 h depending on dope concentration and viscosity to obtain a homogenous dope. The dope was then vacuum de-aerated, tested for viscosity and pH then filtered under pressure through a 25μ cartridge filter before extruding at 2.36 m/min through a 90p/2,000-hole spinneret into a 1.5% (w/w) calcium chloride dihydrate coagulation bath. The as-spun fibres were hauled-off at 2 m/min from the bath, stretched through acetone/water (56/44, v/v) orientation bath, then washed in series of acetone/water baths (Table 10) before finally washing in pure acetone bath and winding onto a drum roller. The fibres were cut off from the drum roller, hand crimped and then dried under extraction before testing for absorbency and available iodine in the fibre.

TABLE 8
Dope composition and solid contents for examples 12 and 13

| | Example 12/ | | Example 13/ | |
|---|---|---|---|---|
| Dope preparation parameters | HG-180730-PVPI | | HG-180214-PVPI | |
| Dope concentration, % w/w | 5 | | 5.5 | |
| Dope composition | Alg. | PVPI | Alg. | PVPI |
| Alg./PVPI concentrations, | 3 | 2 | 5 | 0.5 |
| % w/w in the dope | | | | |
| Expected Alg./PVPI concentrations | 60 | 30 | 90.9 | 9.1 |
| in the fibre | | | | |

| Dope wt., g | 2,500 | 2,000 |
|---|---|---|
| High G Alginate wt., g | 75 | 100 |
| Povidone-iodine (PVPI), wt., g | 50 | 10 |
| Water, wt., g | 2,375 | 1890 |
| Dope pH | — | 5.16 |

TABLE 9
Viscosity of High G alginate-Povidone iodine dopes

| | | Dope composition | | | Spindle speed, rpm/Viscosity, mPa · s or cp | | | | | | | |
|---|---|---|---|---|---|---|---|---|---|---|---|---|
| | | % | | | (Spindle size 04; temperature 22° C.) | | | | | | | |
| Example | Batch | HGAlg | PVP-I | Water | 0.5 | 1.0 | 2.5 | 5 | 10 | 20 | 50 | 100 |
| 12 | HG-180730-PVPI | 3 | 2 | 95 | 800 | 800 | 800 | 800 | 780 | 770 | 740 | 716 |
| 13 | HG-180214-PVPI | 5 | 0.5 | 94.5 | 9,200 | 8,600 | 8,400 | 8,360 | 8,140 | 7,870 | — | — |

TABLE 10
Extrusion conditions for examples 12 and 13

| Example/ | Amount of CaCl₂•2H₂O | Extrusion rate, | Haul off speed, | | | | |
|---|---|---|---|---|---|---|---|
| Batch no. | in the coag. Bath, % | m/min | m/min | Draw speed, m/min | | | |
| Example | Dope viscosity too low for extrusion. To enable extrusion the dope must contain enough | | | | | | |
| 12/ | alginate for spinnable dope viscosity above 2,000 cp. | | | | | | |
| HG- | | | | | | | |
| 180730- | | | | | | | |
| PVP-I | | | | | | | |
| | | | | Draw speed, m/min | | | |
| | | | | (washing bath composition: | | | |
| | Amount of CaCl₂•2H₂O | Extrusion rate, | Haul off speed, | Acetone/water, v/v) | | | |
| | in the coag. Bath, % | m/min | m/min | D1 | D2 | D3 | Winder |
| Example | 1.5 | 2.36 | 2.01 | 3.04 | 3.09 | 3.16 | 3.23 |
| 13/ | | | water | 25/75 | 25/75 | 69/31 | 100/0 |

| | |
|---|---|
| HG- | High jet stretch ratio (0.85) and high draw ratio (1.51). |
| 180214- | Extrusion was carried out under typical conditions used for extruding alginate fibres except |
| PVP-I | for lower acetone contents in the washing baths. Fibres were very heavily coloured (yellow) |
| | as formed in the coagulation bath but progressively lost colour through the acetone/water |
| | washing baths with most colour lost occurring in the high acetone content baths. Lowering |
| | the acetone contents in the baths reduced the rate but did not stop the leaching out of iodine |
| | alone or combination of iodine/PVP-I. The leaching out of iodine occurred in all the baths |
| | including the coagulation bath which became coloured as extrusion progressed. The fibres |
| | were not highly swollen but were not properly separated after coming out of pure acetone |
| | baths. They very were very harsh to handle and difficult to dry presumably due to high |
| | retention of the PVP polymer in the fibre. |

TABLE 11
Fluid absorbency and retention properties
of HG-180214-PVP-I (Example 13) fibres.

| | |
|---|---|
| Solution A | Saline |

| Fluid handling property | 1 min | 30 min | 1 min | 30 min |
|---|---|---|---|---|
| Absorbency, g/g | 11.01 | 17.52 | 19.83 | 21.92 |
| Retention, % | 59.3 | 47.0 | 44.3 | 54 |

As Table 11 appear to show, the fibres produced had good absorbency for high G calcium alginate fibres but low retention which appear to reflect to some extent the effect of PVP in the fibre as observed earlier in examples 6 and 7.

TABLE 12
Available iodine content and possible antimicrobial efficacy of the fibres

| | Available Iodine in the fibre | | | | | |
|---|---|---|---|---|---|---|
| | after extraction in solution A | | Antimicrobial efficacy test (average log reduction after 24 h) | | | |
| | Concept Life sciences | | Test by Surgical Material Testing | | | |
| | company, Bradford: | | Laboratory (SMTL) using the | | Test according to modified ASTM E2149- | |
| | Inductively coupled (or | | ‘Shaking method’ and initial | | 13a using initial inoculum range of 1.5- | |
| | atomic emission | | inoculum range of 1-5.0 × 10⁶ CFU | | 3.0 × 10⁶ CFUmL⁻¹ | |
| Example/ | spectroscopy, ICP-OES | Inhouse | | Klebsiella | | |
| Batch | technique (also called | Titration | Staphylococcus | pneumoniae | Staphylococcus aureus | Klebsiella |
| number | IPC-AES) | technique | aureus bacteria | bacteria | bacteria | pneumoniae bacteria |
| HG-180214- | 0.26 | <0.10 | No reduction rather | No reduction | No reduction | No reduction |
| PVP-I | | | indicated bacteria | rather | | |
| (Example | | | growth | indicated | | |
| 13) | | | | bacteria | | |
| | | | | growth | | |

As expected, the fibre contained very low amount of iodine after all the washing processes and therefore no inhibition observed.

Some of the main conclusions from this investigation include:
No problem with preparation of high G sodium alginate/povidone-iodine solutions but dopes must contain enough alginate (>3% w/w) to obtain spinnable solutions.
Dope degassing was difficult due to excessive bubbles created in the dope and the dark colour of the dope due to the presence of PVP-I. However, degassing was achieved through slow vacuum deaeration and then allowing to stand in the chamber to de-vacuum slowly overnight after achieving the required vacuum pressure (−0.8 to −1.0 bar). The process resulted in increased dope viscosity due to loss of solvent (water) and mostly likely also iodine, both in vapour.
Filtration was easy and carried out using a short (˜6 cm long) 25μ MicraMesh cartridge filter (MMT 2564 25), provided the dope was mixed properly and homogeneous.
Coagulant was as usual calcium chloride dihydrate and at 1.5% concentration
The fibres as spun in the coagulation bath were swollen and this made it easier for PVP-I to leach out of the fibres into the bath. But the problem was expected to be less with High G alginate than with High M alginate with or without psyllium.
PVP-I retention in the fibres was problematic but appeared to improve within a batch in coagulation and washing baths as extrusion progressed. Fibres spun at the beginning of every batch were off white to pale yellow but with the yellow colour deepening at the end of the batch. Iodine leaching was reduced as the washing baths became saturated with iodine and more tows were wrapped on final winding roller.
Retention appeared to improve also with reduced acetone concentrations in the acetone/water baths; except that the fibres became more difficult to dry and stuck together more.
The effect on absorbency of addition PVP-iodine complex in the fibre was remarkable. There was an overall increase in absorbency for the fibres, although the results obtained did not appear to show clearly that increasing concentration of iodine (shown by depth of fibre colour) led to better fibre absorbency. In any case, the work appeared to confirm that PVP-I like PVP without iodine can be used as a substitute for CMC, pectin or psyllium in boosting alginate absorbency.
Unfortunately, the fluid retentions for the samples were rather medium and had not really improved. This high swelling and low retention were the reasons for the leaching out of PVPI from the fibres during extrusion.
As expected, there was no problem with extracting the iodine from the fibres by soaking in aqueous solutions and allowing to stand over time, although a complete extraction was not possible. As the analyses indicated, the level of iodine in the fibres were low (<0.2%) and therefore not enough to inhibit bacteria growth.

### Examples 14-17

High M Alginate-PVPI fibres

These examples describe the preparation of spinnable High M sodium alginate-povidone iodine (PVPI) complex solutions using povidone-iodine supplied by Acros or BASF and followed by initial attempts to prepare High M calcium-PVP-I complex fibres from those solutions that can be spun.

Table 13 details the concentrations and amounts of materials used. The solutions were prepared and de-aerated as described in examples 12 and 13 except that in the present examples, High M sodium alginate powder (Manucol DH supplied by FMC Biopolymers, UK, Ltd) and PVP-I powder (30/06 supplied by BASF) or PVP-I powder supplied by Acros were used.

TABLE 13
Dope composition and solid contents for examples 14-18

| | Example 14/ | | Example 15/ | | Example 16/ | | | | | |
|---|---|---|---|---|---|---|---|---|---|---|
| | HM- | | HM- | | HM- | | Example 17/ | | Example 18/ | |
| | 180124- | | 180125- | | 180730- | | HM- | | HM/HG- | |
| | PVPI | | PVPI | | PVPI | | 180207-PVPI | | 180807-PVPI | |
| Dope preparation parameters | (Acrōs PVP-I) | | (Acrōs PVP-I) | | (Acrōs PVP-I) | | (BASE PVPI) | | (BASF PVPI) | |
| Dope concn, % w/w | 21.12 | | 13.5 | | 8.4 | | 5.5 | | 6.0 | |
| Dope composition | Alg. | PVPI | Alg. | PVPI | Alg. | PVPI | Alg. | PVPI | HM/HG | PVPI |
| | | | | | | | | | Alg | |
| Alg./PVPI concns, % w/w in dope | 5.34 | 15.78 | 5.4 | 8.1 | 5 | 3.4 | 5 | 0.5 | 4.05/ | 1.5 |
| | | | | | | | | | 0.45 | |
| Expected Alg./PVPI concns in the fibre | 25.3 | 74.7 | 40.0 | 60.0 | 59.5 | 40.5 | 90.9 | 9.1 | 67.5/ | 25 |
| | | | | | | | | | 7.5 | |

| | | | | | |
|---|---|---|---|---|---|
| Dope wt., g | 380.3 | 370.0 | 2,190 | 2,000 | 6,000 |
| High M Alginate wt., g | 20.3 | 20 | 109.5 | 100 | 270 |
| Povidone-iodine (PVPI), wt., g | 60 | 30 | 74.5 | 10 | 90 |
| Water, wt., g | 300 | 320 | 2006 | 1890 | 5,640 |
| Dope pH | — | — | 4.23 | 4.87 | |

TABLE 14
Viscosity and spinnability assessments of High M sodium alginate-Povidone iodine complex dopes

| | | Dope composition | | | Spindle speed, rpm/Viscosity, mPa · s or cp | | | | | | | |
|---|---|---|---|---|---|---|---|---|---|---|---|---|
| | | % | | | (Spindle size 04; ambient 19-21° C.) | | | | | | | |
| Example | Batch | HM Alg | PVP-I | water | 0.5 | 1.0 | 2.5 | 5 | 10 | 20 | 50 | 100 |

| 14 | HM- | 5.3 | 15.8 | 78.9 | 332,000 | Viscosity too high to be measured. | | | | | |
|---|---|---|---|---|---|---|---|---|---|---|---|
| | 180124- | | | | | Solution appeared very dark (dark-brown) and almost static | | | | | |
| | PVPI | | | | | Too viscous to flow, pump and filter to spin but when pulled using a spatula, it | | | | | |
| | (Acrōs | | | | | flowed well from it indicating a possibility of extrusion perhaps by a gel-spinning | | | | | |
| | PVP-I) | | | | | technique. | | | | | |
| 15 | HM- | 5.4 | 8.1 | 86.5 | 208,000 | 177,400 | Viscous still too high and could not be measured at any higher shear | | | | |
| | 180125- | | | | | | speed than 1.0 rpm | | | | |
| | PVPI | | | | | | Improved flow rate but still too viscous and difficult to deaerate and | | | | |
| | (Acrōs | | | | | | filter for extrusion | | | | |
| | PVP-I) | | | | | | | | | | |
| 16 | HM- | 5.0 | 3.4 | 91.4 | 20,400 | 19,000 | 16,240 | 14,200 | 12,300 | Spinnable viscosity with satisfactory | |
| | (Acrōs | | | | | | | | | flow. | |
| | PVP-I) | | | | 37,600 | 31,200 | 24,320 | 19,760 | 16,100 | Viscosity after standing for 4 days | |
| | 180730- | | | | Dope appeared to change with time. It thickened after allowing to stand for 4 days and did not | | | | | | |
| | PVPI | | | | flow readily unstirred but thinned out on shearing with improved flow. | | | | | | |
| 17 | HM- | 5.0 | 0.5 | 94.5 | 9,400 | 8,700 | 8,280 | 8,160 | 7,670 | 7,095 | As prepared before |
| | PVPI | | | | | | | | | | vacuum deaeration |
| | (BASF | | | | 14,200 | 13,100 | 11,840 | 10,980 | 10,110 | 8.890 | After vacuum |
| | PVPI) | | | | | | | | | | deaeration |
| | 180207- | | | | | | | | | | |
| 18 | HM/HG- | 4.5 | 1.5 | 94 | 17,600 | 14,800 | 12,400 | 10,720 | 9,360 | 7,940 | After deaeration |
| | 180807- | HM/HG | | | | | | | | | |
| | PVPI | | | | | | | | | | |
| | (BASF | | | | | | | | | | |
| | PVPI) | | | | | | | | | | |

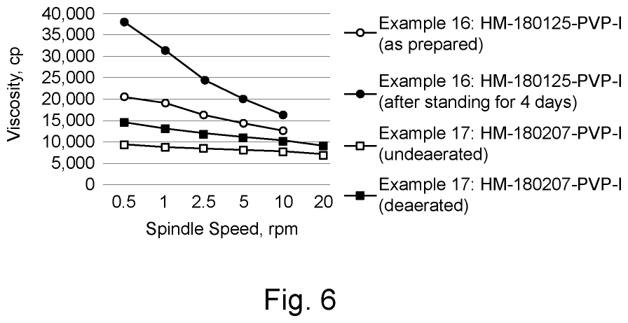

The spinnable dopes were each vacuum de-aerated before extrusion but with extra care for the higher content PVP-I dope (Example 16). This dope was extremely difficult to vacuum deaerate due to excessive foam generated. Deaeration was carried out much slower and in stages before finally leaving to stand overnight to de-vacuum slowly. Each dope was filtered and spun as in examples 12 and 13 except for the spinning conditions shown in Table 15. The viscosity-shear speed profile of High M alginate-PVP-I dopes according to examples 16 and 17 are shown in FIG. 6.

TABLE 15
Extrusion conditions for examples 16 and 17

| | Spinning conditions | | | Washing baths: | |
|---|---|---|---|---|---|
| | | | Haul-off/ | (Acetone/H2O, | |
| | | | D1/D2/D3/D4/ | % v/v) | |
| | Coagulant | Pump | winder | Ace/H2O bath 1 | |
| Example/ | 1st/2nd coagulation | speed | (all speeds | Ace/H2O bath 2 | |
| Batch No. | baths | (rpm) | in m/min) | Ace/H2O bath 3 | Comments |
| 16/ | 1.5% CaCl₂•2H₂O/ | 6.0 | 6.53/8.11/ | 50/50 . . . 1 | Various dope deaeration methods |
| HM-180125- | 1% CaCl₂•2H₂O | | 8.66/8.55/ | 80/20 . . . 2 | tried but vacuum appeared most |
| PVPI- | | | 8.59 | 100/0 . . . 3 | successful. Initial trials showed PVP- |
| (Acros) | | | (spinneret | | I stripped off the fibre in coag and |
| | | | 90μ/30 holes) | | washing baths especially baths with |
| | | | | | high acetone concentrations. |
| | | | | | Established washing procedure for |
| | | | | | extruding calcium alginate and high |
| | | | | | spinning speeds through the baths |
| | | | | | did not reduce the PVP-I leaching |
| | | | | | out of the fibre. Fibres stuck |
| | | | | | together and inseparable after |
| | | | | | drying. |
| 17/ | Used coagulant from | 8.2 | 2.01/3.14/ | 40/60 - - - 1 | Acetone concentrations in the |
| HM-180207- | previous extrusion but | | 3.21/3.21/ | 56/44 - - - 2 | washing baths reduced. Fibres |
| PVPI | balanced with addition | | 3.15 | 73/27 - - - 3 | produced in the first 15 mins showed |
| (BASF) | of 3 litres of a 2% | | (spinneret | | only very pale-yellow colour, but the |
| | CaCl₂•2H₂O soln. | | 90μ/2000 holes). | | colour deepened slightly as |
| | | | | | extrusion progressed. |
| | | 14.4 | 1.36/1.50/ | 56/44 - - - 1 | Usual problems with iodine |
| | | | 1.55/1.56/ | 70/30 - - - 2 | retention in the fibres, (note: Haul- |
| | | | 1.55 | 77/23 - - - 3 | off bath (2nd coag) contained |
| | | | (spinneret | Winder bath: | ace/water, 40/60) |
| | | | 90μ/2000 holes) | 100% acetone | |

| | | | |
|---|---|---|---|
| 18/ | Used coagulant from | Spinning conditions as above but no washings at all except | Fibres were mangled as soon as out |
| HM/HG- | previous extrusion but | in the winder bath containing 3191.3 g acetone/water/PVPI | of the coag. bath and before winding |
| 180807- | balanced with fresh 3 | (94%/5.83%/0.17%) mixture. PVPI colour faded as soon as | to squeeze out excess coagulant. |
| PVPI | litres CaCl₂•2H₂O soln | solution mixed with acetone. | Fibre had very deep dark yellow |
| (BASF) | (1.5%). | | colour before winder bath but colour |
| | | | gradually faded as soon washed in |
| | | | the acetone/water/PVPI bath. Fibres |
| | | | were difficult to dry and separate but |
| | | | separation and softness improved by |
| | | | spraying partially dried fibre with |
| | | | propylene glycol, PG/Tween 20 |
| | | | solution (1:1 ratio) before drying |

Spinneret: 90μ/2000 holes; Filtration system: 25μ cartridge (short); Pump capacity 2.4 cc/rev; D1, D2 and D3 drawing rollers

TABLE 16
Fluid absorbency and retention properties of HM-180125-PVP-I
(Example 16) and HM-180207-PVP-I (example 17) fibres.

| | Absorbency, g/g | | | |
|---|---|---|---|---|
| | (Retention, %) | | | |
| Example/ | Solution A | | Saline | |
| Batch No. | 1 min | 30 min | 1 min | 30 min |
| 16/ | 7.9 | 6.8 | 22.8 | 26.4 |
| HM-180125-PVPI | (58.3%) | (69.7%) | (79.7%) | (79.0%) |
| (Acrōs) | | | | |
| 17/ | 29.3 g/g | 28.2 g/g | 31.8 g/g | 35.9 g/g |
| HM-180207-PVPI | (70.3%) | (64.0%) | (77.7%) | (62.7 g/g) |
| (BASF) | | | | |

Some of the conclusions from this investigation include:
All the comments made in examples 12 and 13 for extruding High G alginate-PVPI fibres are applicable to High M alg.-PVPI system.
However, extrusion of High M alg.-PVPI fibres was found to be more difficult as the as-spun fibres in the coagulation and washing baths were more highly swollen/sticky, making it more difficult to handle and dry the fibres and much easier for iodine to leach out of the fibres into the baths-from a visual comparison of the depth of iodine colour in the fibres for High G alg.-PVPI relative to those of High M alg.-PVPI.
PVP-I retention in the fibres was problematic but appeared to improve within a batch in coagulation and washing baths as extrusion progressed. So, fibres spun at the beginning of every batch was almost colourless to pale yellow with the yellow colour deepening at the end of the batch. PVP-I retention appeared to improve also with reduced acetone concentrations in the acetone/water baths; except that the fibres became more difficult to dry and stuck together more.
The effect on absorbency of PVP-iodine in the fibre was even more remarkable for High M alginate as shown in Table 16 when compared with High G-PVPI fibres especially in saline. This is perhaps not surprising; as generally, high M alginates absorb and retain liquid more than high G, provided the fibres are properly opened.
The low results obtained in solution A for example 16 was due to unopened fibres. As indicated in table 15, some difficulties were experienced with the production of this batch.
Exempting example 16 in solution A, all the sample had very fast rate of absorbency and gelation and gels formed were very soft. The fluid retentions which had always been in the low to medium range were high above 60%.

### Examples 18 and 19

### High M Alginate-Psyllium-PVPI Fibres

This example describes the extraction of psyllium gel from psyllium seeds and production of calcium alginate—psyllium—PVPI fibres. The psyllium gel was prepared by stirring 205 g of psyllium seeds (supplied by ‘Natural Health 4 Life’ in Devon, www.naturalhealth4life.co.uk) into 5000 g of water (at 80-100° C.). contained in F-20 L Single Layer Glass Reactor (obtained from Zhengzhou Keda machinery & instrument equipment co., Ltd). After addition, stirring continued at 60 rpm for 35 minutes; then 100 g sodium hydroxide solution (5% w/w) was added and stirring increased to 100 rpm for 2 minutes before heating and stirring were stopped to allow the extracted seeds to settle at the bottom of reactor. After standing for 15 minutes and temperature still above 85° C., the reactor discharge valve was opened, first to expel the extracted seeds for disposal, then the psyllium gel (4000 g, pH˜12) decanted through ˜60μ sieve if necessary, into a 10-litre plastic container. At this temperature, one decanting process was enough to separate most of the dissolved mucilage from the seeds as the gel flowed well due to low viscosity. But at lower temperatures (<85° C.), addition of further NaOH solution to reduce viscosity and followed by successive decanting processes were necessary before almost all the gel could be separated from the seeds.

The hot psyllium gel was left to cool (40° C. to ambient), then mixed well with a high shear mixer (at 2500−3500 rpm) until thin and uniform. The gel pH between 11 and 12 (determined using Hanna HI 211 pH/ORP meter) was lowered to between 5 and 6 by addition of 1M HCl.

The alginate-psyllium-PVPI dope was prepared by dissolving 3-5% w/w (typically 60-100 g) alginate (high M or high G or mixture) containing the required amount of PVPI powder (˜0.5% w/w; 10 g) into the psyllium solution (typically 2,000 g; concentration 0.8-1% w/w) using a high shear mixer at 4,000-6,000 rpm for 1-2 h depending on viscosity and solid content. The dope was then transferred into a dope reservoir, vacuum deaerated overnight and spun after viscosity assessment following spinning conditions shown in Table 17.

TABLE 17
Effect on viscosity of addition of psyllium to PVP-I/alginate dopes

| | Dope Composition | | | | Spindle speed, rpm/Viscosity, mPa · s or cp | | | | |
|---|---|---|---|---|---|---|---|---|---|
| | (%) | | | | (Spindle size 02; temperature 17° C.) | | | | |
| Example/Batch No. | PVP-I | Psyllium | Alg. | water | 0.5 | 1.0 | 2.5 | 5 | 10 |
| 18/ | 0.46 | 0.79 | 4.13 | 93.85 | 46,400 | 38,800 | 29,360 | 24,560 | 19,860 |
| HM-180212-P-PVP-I | | | | | Before deaeration | | | | |
| | | | | | 50,400 | 40,000 | 30,400 | 24,880 | |
| | | | | | After deaeration (dope pH 4.93) | | | | |
| 19/ | 0.48 | 0.65 | 4.77 | 94.11 | 30,800 | 28,800 | 21,600 | 17,640 | 14,740 |

| | |
|---|---|
| HG-180216-P-PVP-I | After deaeration (dope pH 5.16) |

Note:
(1) 30/06 PVP-I obtained from BASF
(2) Deaeration achieved by slow vacuuming, then keeping under vacuum (−0.45 bar) overnight
Extrusion conditions for examples 18 and 19

| | | | | Washing baths | |
|---|---|---|---|---|---|
| | Spinning conditions | | | Acetone/H₂O | |
| | | Pump | Haul-off/D1/D2/D3/ | Ace/H₂O bath 1 | |
| | Coagulation | speed | winder | Ace/H₂O bath 2 | |
| Example/Batch No. | bath | (rpm) | (all speeds in m/min) | Ace/H₂O bath 3 | Comments |
| 18/ | Used | 8.2-8.8 | 2.01/3.14/3.21/3.21/3.15 | 25/75-1 | Fibre coagulation appeared slower and |
| HM-180212-P-PVP-I | coagulant | | | 44/56-2 | required extra addition of CaCl₂•2H₂O. |
| | from previous | | | 69/31-3 | Fibres appeared also more swollen in |
| | extrusion but | | | PVPI still | bath because of psyllium and PVP-I |
| | balanced with | | | leached out | leaching into coagulation and ace/water |
| | addition of | | | | baths was worse. Fibres, very faintly |
| | 82 g of solid | | | | coloured |
| | CaCl₂•2H₂O | | | | |
| | to bath to | | | | |
| | enable | | | | |
| | extrusion. | | | | |
| 19/ | Used 1.5% | 12.1 rpm | 3.08/3.78/3.80/3.81/3.78 | 65/35-1 | Effects on extrusion and PVP-I |
| HG-180216-P-PVP-I | CaCl₂•2H₂O | (but | 3.08/3.781/D2/D3/3.78 | 51/49-2 | retention in fibres were as in example |
| | soln. | 7.1 rpm | D1 and D2 omitted | 73/27-3 | HM-180212-P-PVPI above although |
| | | initially) | | | retention of PVPI in high alginates |
| | | | | | were slightly better as the fibres |
| | | | | | swelled less in coagulant and washing |
| | | | | | baths. |

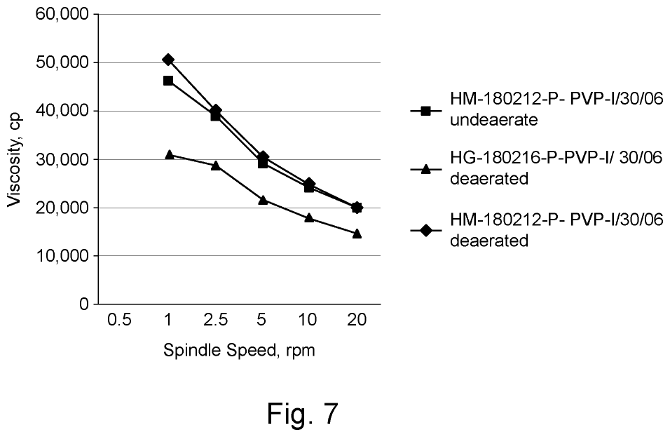

Some of the conclusions from this investigation include:
Generally, the depth of colour of PVPI in alginates dopes containing psyllium appeared deeper than observed for dopes with no psyllium, indicating that psyllium may contain groups that enhance iodine colour.
Dope viscosities (Table 18) especially for High M alg./psyllium/PVPI were potentially very high after deaeration at the concentrations examined but they were spun. The recommendation is to use only 3-4% w/w alginate concentration in the dope as higher concentrations would produce dopes too difficult to mix, deaerate and filter. The results are shown in FIG. 7 which is a graph of the viscosity-shear speed profile of the effect of addition of psyllium in alginate/PVP-I dopes.
It did not appear that addition of PVPI to a psyllium-alginate system added much advantage apart from perhaps consistency in fluid absorbency. The fibres were difficult to extrude and dry due to excessive swelling in coagulation and washing baths.
Consequently, as expected, the fibres produced were only very slightly coloured. Almost all the PVP-I colour had disappeared from the fibres by the end of each extrusion.
Fluid absorbency (Table 19) in solution A and saline averaged at about 27 g/g for either 1 min or 30 minutes immersion for high M/psyllium/PVPI fibres although the range was between 26 and 34 g/g except for the unexpected low result of 19 g/g in saline for 1 minute which was ascribed to test error and/or problems with the sample tested. All the fibres formed soft and integral gels in both liquids with fluid retention varying between 69 and 79%. These results are like those obtained in earlier examples for high G alginate/PVP-I fibres without psyllium.
Fluid absorbency (10-19 g/g) and retention (54-92%) results for high G/psyllium/PVP-I fibres were also similar and in cases lower than the results obtained in examples 12 and 13 for high G/PVPI fibres without psyllium. The low results and inconsistencies obtained here in the present examples were due to difficulties in fibre preparations which might led to much variations in the fibres samples.

TABLE 18
Fluid absorbency and retention properties of alginate/psyllium/PVP-I
fibres (examples 18 and 19)

| | Absorbency, g/g | | | |
|---|---|---|---|---|
| | (Retention, %) | | | |
| Example/ | Solution A | | Saline | |
| Batch No. | 1 min | 30 min | 1 min | 30 min |
| 18/ | Range 26-31 | Range 26-31 | Range 19-34 | Range 26-33 |
| HM-180212- | 26.4 | 26.8 | 26.4 | 27.9 |
| P-PVP-I | (69.3%) | (71.3%) | (73.0%) | (79.0%) |
| 19/ | 9.9 g | 13.7 g/g | 12.8 | 18.7 g/g |
| HG-180216- | (73%) | (54%) | (92%) | (67%) |
| P-PVP-I | | | | |

### Examples 20 and 24

Alginate-PVPI-Glycol fibres

These examples describe the attempts made to reduce PVP-I leaching out of fibres during extrusion. The process as indicated in Tables 19-21 involved the modification of Alg./PVI dopes by the addition of non-toxic and easily solvent miscible diols such as propylene glycol (PG, 4-10%) or polyols such as polyethylene glycol (PEG, 0.5-12%) followed by coagulation in 1.5% aqueous calcium chloride solution or in a bath containing˜60% iso-propanol (IPA)/38% water/1.4% calcium chloride/1.3% PEG or in a bath containing 60% water/37% iso-propanol (IPA)/1.5% calcium chloride/1.3% PEG,. Then a post-coagulation treatment was applied using any of the following steps:
Fibres mangled before haul-off roller to squeeze out excess coagulant and further mangled after drawing (between first draw roller D1 and the second draw roller D2 in
Fibres spun without washing except at the winder roller bath that contained pure acetone or acetone with Tween 20 or acetone/PG/water.
Fibres washed only at winder bath consisting of 97.8% IPA/1.2% PVPI/0.8% water/0.2% PEG/
Fibre after spinning was left on the winder roller under extraction system overnight.

The fibres were then dried under low extraction systems after excess liquid was squeezed out. Typically, a batch was prepared from a dope weight of 5,000-6,000 g containing 4.5-6% w/w solids. Dope preparation was as described in examples 12 and 13 except that in the present examples the required solids were first weighed separately, then mixed together properly in dry states before gradually adding to water that was being vigorously stirred with a high shear mixer until all the solids were completely dissolved. This was followed whilst still mixing continuously by a gradual addition of the required amount of PG or PEG and a further mixing for at least 30 minutes to ensure a homogenous dope.

TABLE 20
Dope concentrations and compositions for examples 20-24

| | | Dope | Dope composition % w/w | | | | | |
|---|---|---|---|---|---|---|---|---|
| | Dope | solid | Alginate | | | | | |
| Example/ | wt. | content | High | High | | | | |
| Batch No. | g | % w/w | M | G | Water | PVPI | PG | PEG |
| 20/ | 6,000 | 6.00 | 4.0 | 0.5 | 90 | 1.5 | 4.0 | — |
| HM/HG- | | | | | | | | |
| 180831- | | | | | | | | |
| PVPI/PG | | | | | | | | |
| 21/ | 6,000 | 6.00 | 4.0 | 0.5 | 84 | 1.5 | 10.0 | — |
| HM/HG- | | | | | | | | |
| 180910- | | | | | | | | |
| PVPI/PG | | | | | | | | |
| 22/HM- | 5,372 | 5.84 | 3.7 | — | 83 | 2.1 | — | 11.2 |
| 181024- | | | | | | | | |
| PVPI/PEG | | | | | | | | |
| 23/HM- | 6,000 | 4.50 | 3.5 | — | 95 | 1.0 | — | 0.5 |
| 181116- | | | | | | | | |
| PVPI/PEG | | | | | | | | |
| 24/HG- | 5,000 | 5.00 | — | 4.00 | 94.5 | 1.0 | — | 0.5 |
| 181204- | | | | | | | | |
| PVPI/PEG | | | | | | | | |

TABLE 21
Effect of addition of PG or PEG on viscosities of a selected alginate/PVPI dopes.

| | Dope Composition | | | | | Spindle speed, rpm/Viscosity, mPa · s or cp | | | | |
|---|---|---|---|---|---|---|---|---|---|---|
| Example/ | (%) | | | | | (Spindle size 04; temperature 21° C.) | | | | |
| Batch No. | Water | Alg. | PVPI | PG | PEG | 0.5 | 1.0 | 2.5 | 5.0 | 10.0 |
| 21/ | 84 | 4.5 | 1.5 | 10 | — | 14,000 | 14,000 | 12,640 | 11,360 | 10,080 |
| HM/HG- | | | | | | | | | | |
| 180910- | | | | | | | | | | |
| PVPI/PG | | | | | | | | | | |
| 22/ | 83 | 3.7 | 2.1 | — | 11.2 | 21,200 | 20,800 | 17,200 | 13,880 | 10,620 |
| HM- | | | | | | | | | | |
| 181024- | | | | | | | | | | |
| PVPI/PEG | | | | | | | | | | |
| 23/ | 95 | 3.5 | 1.0 | — | 0.5 | 3,200 | 3,200 | 3,120 | 3,080 | 2,920 |
| HM- | | | | | | | | | | |
| 181116- | | | | | | | | | | |
| PVPI/PEG | | | | | | | | | | |

Each dope prepared was as usual vacuum de-aerated, tested for viscosity where necessary (Table 21) and then spun after filtration through a 90μ/2,000-hole spinneret using conditions set out for each example in Table 22. The fibres were cut off from the drum roller, hand crimped and then dried under extraction before testing for absorbency and available iodine in the fibre.

TABLE 22
Extrusion conditions for examples 20 to 24

| | Spinning conditions | | | Washing baths | |
|---|---|---|---|---|---|
| | | | Haul-off/ | Bath 1 for D1 | |
| | | Pump | D1/D2/D3/ | Bath 2 for D2 | |
| Example/ | Coagulation | speed | winder | Bath 3 for D3 | |
| Batch No. | baths | (rpm) | m/min) | Winder bath | Comments |
| 20/ | Used coagulant | 9.6 | 1.2/1.74/ | All baths empty | Only one wrap of fibre on each roller. |
| (note: | (aq. 1.5% | | 1.82/1.86/ | except winder | As-spun fibres were laden with PVPI and |
| dope has | CaCl₂•2H₂O soln | | 1.83 | bath containing | very dark. Fibres wrapped on the winder |
| 4% PG) | with PVPI from | | | pure acetone | roller. Only fibres on top layer and in |
| HM/HG- | previous | | | | contact with acetone were stripped of |
| 180831- | extrusion) freshen | | | | PVPI. The bottom layers were still rich |
| PVPI/PG/1 | with new 8-litres | | | | with PVPI. |
| | of 1.5% Aq. | | | | But fibres were still harsh after drying |
| | CaCl₂•2H₂O soln. | | | | though at reduced level than without PG in |
| | | | | | the dope. |
| HM/HG- | | | 1.2/1.74/ | All baths empty | As for in HM/HG-180831-PVPI/PG/l |
| 180831- | | | 1.82/1.86/ | except winder | except that fibres were sprayed with |
| PVPI/PG/2 | | | 1.83 | bath containing | Tween 20 dissolved in acetone to increase |
| | | | | pure acetone | fibre softness and promote separation |
| HM/HG- | Used coagulant | 9.6 | 1.41/1.86/ | Fibres mangled | Fibres wound on the winder roller and left |
| 180831- | (aq. 1.5% | | 1.91/1.94/ | before haul-off | to dry under the extraction system |
| PVPI/PG/3 | CaCl₂•2H₂O soln | | 1.90 | and D2 rollers | overnight. Outer layer of fibres slightly |
| | with PVPI from | | | Haul-off bath - | over dried and harsh but harshness |
| | previous | | | acetone/1% | reduced when sprayed with |
| | extrusion) freshen | | | Tween 20 | acetone/water/Tween 20 mixture. Fibres |
| | with new 8-litres | | | Bath 3 - acetone | cut into staple and opened partially |
| | of 1.5% Aq. | | | | damped with a mini-carder. Fibres well |
| | CaCl₂•2H₂O soln. | | | | opened and soft handle |
| HM/HG- | | | .41/1.86/ | Fibres mangled | Fibres dried partially in the extraction |
| 180831- | | | 1.91/1.94/ | before haul-off | system. No significant change noticed |
| PVPI/PG/4 | | | 1.90 | and D2 rollers | except the usual problem of PVPI leaching |
| | | | | All baths empty | in pure acetone. |
| | | | | except winder | |
| | | | | bath containing | |
| | | | | pure acetone | |
| 21/ | Fresh 1.5% | 9.6 | 1.41/1.86/ | Fibres mangled | Fibres wound and left to dry under the |
| (note: | CaCl₂•2H₂O soln. | | 1.91/1.94/ | before haul-off | extraction system overnight, then cut and |
| dope has | | | 1.84 | and D2 rollers. | opened using a mini-carder whilst still |
| 10% PG) | | | | All baths empty | damp |
| HM/HG- | | | | except winder | Fibres had deeper iodine colour |
| 180910- | | | | bath containing | Less leaching into coagulation bath |
| PVPI/PG/1 | | | | pure acetone | Less leaching into acetone bath |
| | | | | | But fibre drying became more difficult |
| 21/ | IPA/Water/calcium | 10.0 | 2.0/2.6/ | Fibres mangled | Higher IPA concentration (>70%) in the |
| HM/HG- | chloride: −1.4% | | 2.7/2.7/ | before haul-off | coag. bath resulted in excessive leaching |
| 180910- | (90 g) | | 2.6 | and D2 rollers | of PVPI from the fibres |
| PVPI/PG/2 | CaCl₂•2H₂O in | | | All baths empty | Fibres were lifted out of the bath as soon |
| | 38.2% (2.5 kg) | | | except winder | as coagulated |
| | water mixed with | | | bath containing | Only 1 wrap on each roller except winder |
| | 60.4% (3.95 kg) | | | pure acetone | where fibres were wound up |
| | isopropanol (IPA) | | | | Fibres were pale yellow on emerging |
| | | | | | from bath and storing on winder. But the |
| | | | | | colour deepened to dark brown on |
| | | | | | standing |
| 22/ | Fresh 1.5% | 9.5 | 1.41/1.86/ | Fibres mangled | Extrusion was excellent |
| (note: | CaCl₂•2H₂O soln. | | 1.91/1.94/ | before haul-off | PVPI leaching from fibres greatly reduced |
| dope has | | | 1.90 | and D2 rollers | in the coagulant and acetone/water baths |
| 11.2% PEG) | | | | No washings at | Fibres were appeared very dark when |
| HM-181024-P | | | | haul-off, D3 and | wound up on the winder roller |
| VPI/PEG/1 | | | | winder baths | The fibres were much softer when damp |
| | | | | Fibres washed at | than with PG but still harsh if completely |
| | | | | D1 (ace/H₂O, | dried |
| | | | | 60/40) and D2 | Overall, excellent process for producing |
| | | | | (ace/H₂O, 35/75) | alg./PVI fibres |
| 22/ | 1.5% CaCl₂•2H₂O | 9.5 | 1.41/1.86/ | Fibres mangled | Attempted drying under extraction system |
| HM-181024- | soln from HM- | | 1.91/1.94/ | before haul-off | but failed to dry properly. |
| PVPI/PEG/2 | 181024-PVPI/PEG/l | | 1.90 | and D2 rollers | Fibres appeared to have swelled and |
| | | | | Washings: | retained too much water |
| | | | | Haul-off bath (2 | In future, no washing in water at all as |
| | | | | warps on roller | attempts produced fibres that swelled too |
| | | | | and washed in | much and became very difficult to dry |
| | | | | water)/D1, D2 | |
| | | | | & D3 baths (1-2 | |
| | | | | wraps, ace/H2O | |
| | | | | wash) | |
| | | | | Winder roller - | |
| | | | | No washing | |
| 23 | Coagulant | 12.7 | 1.45/2.10/ | Fibres mangled | PVPI leaching from fibres were very |
| (note: | composition of: 1.5% | (Extrn | 2.14/2.16/ | before haul-off | greatly reduced in the coagulation and |
| dope has | CaCl₂•2H₂O | rate | 2.16 | and D2 rollers | winder baths |
| 0.5% PEG | dissolved in | 2.39 m/ | | No washings at | Fibres could be squeezed out without any |
| HM-181116- | 60% water/37% | min) | | all at the Haul- | substantial amount of PVPI being |
| PVPI/PEG | IPA/1.3% PEG | | | off, D1, D2 and | squeezed out |
| | | | | D3 baths except | Undried and damp fibres were soft and |
| | | | | at the winder | easily opened but drying completely still |
| | | | | bath which | rendered the fibres stiff, harsh and |
| | | | | contained pure | difficult to open |
| | | | | IPA | An alternative washing arrangement with |
| | | | | | washings at D1 bath (60% H2O/28% |
| | | | | | IPA/1% PEG) and winder bath (pure IPA) |
| | | | | | gave a satisfactory result also. |
| | | | | | Fibres were dried under extraction system |
| | | | | | to a moisture content of 30-45%, then cut |
| | | | | | and opened immediately or stored in a |
| | | | | | sealed bag. Moisture content of 45% was |
| | | | | | best as fibres dried to less moisture were |
| | | | | | harsh and difficult to open |
| 24/ | Fresh 1.5% | 12.1 | 1.45/2.10/ | Fibres mangled | The mangling process squeezed much of |
| HG-181204- | CaCl₂•2H₂O soln. | | 2.14/2.16/ | before haul-off | the coagulant out of the fibre before |
| PVPI/PEG | | | 2.16 | and D2 rollers | reaching the winder |
| | | | | Fibres were | Fibres were dried to required moisture |
| | | | | wound with 4-7 | content (40-45%) under extraction system |
| | | | | wraps on each | Fibres obtained were dark and soft when |
| | | | | roller but | wet but yellow when dried |
| | | | | without | |
| | | | | washings at all | |
| | | | | except at the | |
| | | | | winder bath | |
| | | | | which | |
| | | | | contained: | |
| | | | | 98.8% IPA/ | |
| | | | | 1.2% PVPI/ | |
| | | | | 0.8% H₂O/ | |
| | | | | 0.2% PEG | |

TABLE 23
Fluid absorbency and retention properties of some
selected alginate/PVPI fibres containing PG or PEG

| | Absorbency, g/g | | | |
|---|---|---|---|---|
| | (Retention, %) | | | |
| Example/ | Solution A | | Saline | |
| Batch No. | 1 min | 30 min | 1 min | 30 min |
| 20/ | 22.33 | 23.52 | 26.21 | 26.64 |
| HM/HG-180831-PVPI/PG | (65) | (72) | (80) | (76) |
| 21/ | 17.09 | 18.27 | 17.08 | 19.05 |
| HM/HG-180910-PVPI/PG | (71) | (75) | (75) | (87) |
| 22/ | 23.10 | 23.65 | 25.07 | 23.61 |
| HM-181024-PVPI/PEG | (84) | (79) | (75) | (82) |
| 23/ | 18.42 | 20.74 | 19.45 | 26.40 |
| HM-181116-PVPI/PEG | (85) | (78) | (82) | (87) |
| 24/ | 7.87 | 12.21 | 12.12 | 14.75 |
| HG-181204-PVPI/PEG | (63) | (37) | (55) | (54) |

TABLE 24
Iodine contents of alginate fibres containing PVPI, PG or PEG

| | | Iodine Content | |
|---|---|---|---|
| | PVPI | External | In-house titration |
| | content | results | % Available iodine |
| Example/ | In dope | Total Iodine | (after samples |
| Batch No | (%) | (mg/Kg) | extracted overnight) |
| Commercial product, | N/A | 1600 | 1.0 |
| InadineTM | | | |
| 20/ | 1.5 | Not Tested | 0.9 |
| HM/HG-180831- | (PG 4%) | | |
| PVPI/PG | | | |
| 21/ | 1.5 | 3700 | 0.7 |
| HM/HG-180910- | (PG 10%) | | |
| PVPI/PG | | | |
| 22/ | 2.1 | 1500 | 0.3 |
| HM-181024- | (PEG | | |
| PVPI/PEG | 11.2%) | | |
| 23/ | 1.0 | Not Tested | 0.4 |
| HM-181116- | (PEG 0.5%) | | |
| PVPI/PEG | | | |
| 24/ | 1.0 | Not Tested | 0.4 |
| HG-181204- | (PEG 0.5%) | | |
| PVPI/PEG | | | |

### Production of Alginate-Iodine-Glycol Fibres and Felts

The potential controlling effects of the glycols (PG and PEG) on the ‘leaching-out’ of PVPI from fibres during extrusion and possibly also post extrusion, was also investigated by incorporating non-complex iodine into fibres and felts through:
Extrusion of dopes containing dissolved iodine crystals
Dyeing fibres or non-woven with solutions containing dissolved iodine crystals.

The dope preparations and extrusion were carried out as described in examples 20-24 for alginate-PVPI-glycol fibres except that 0.5% w/w iodine crystals (supplied by Alfa Aesar, mp 183-186° C., mol. wt (or FW) 253.81, density 4.930 g/cm³) were used in place of PVPI. Small amounts of PG or PEG were ground using mortal/pestle before adding to the dope and mixing homogeneously to give very dark dope solutions. Dopes were either vacuum deaerated or sealed and left standing to deaerate overtime. Vacuum deaeration as expected led to rapid escape of iodine from the dope. Extrusion was satisfactory except for partial gelation of dopes.

Fibres produced had deep iodine colour initially, but this faded very quickly on exposure to air and even after sealing, the iodine gradually vaporised out of the fibres leaving it colourless. Addition of PG or PEG retarded evaporation but did not stop it. Similar problems were observed when producing the final materials using a ‘dyeing’ process in place of the extrusion process as described herein.

Some of the conclusions from this investigation include:
Generally, the addition of PG or PEG in dopes, coagulation and washing baths separately or together appeared to increase PVPI retention in the fibre during extrusion. The effect was noticeable irrespective of the amounts added, although the more the better. The only problem was that the fibres became more difficult to dry with increasing amounts.
The dope viscosities obtained in Table 21 showed that the amounts of these materials (PG and PEG) added to the dopes especially PG did not substantially increase their viscosities.
To obtain maximum retention of PVPI, fibres were mangled by passing between two rollers to remove excess coagulant, stretched between successive rollers to impact strength and washed only during wound up at the winder roller bath.
Winder roller washing bath compositions that appeared satisfactory are:
   pure acetone or IPA
   pure acetone containing<5% spin finish such as Tween 20
   pure acetone or IPA containing any amount of PG or PEG
   98.8% IPA/1.2% PVPI/0.8% H₂O/0.2% PEG
Generally, the depth of colour of PVPI in dopes as observed with addition of psyllium and in the fibres (where retained) appeared to deepen with PG and especially PEG.
In order to cut, open and card the fibres produced, they had to be dried to retain a moisture content between 35 and 45%; otherwise, the fibres would become stuck together, harsh to handle and very difficult to open and would not card well.
Fibres spun and washed in water could not be dried adequately due to excessive swelling and water retention and this must be avoided Fibres were highly absorbent especially where high M alginates were used.
The pattern of iodine contents and available iodine in the fibres as displayed in Table 24 were difficult to understand apart from the general understanding that they were affected by way the samples were coagulated, processed and handled during production. But what might also be true was the effect of type of glycol used. It appeared that PEG has a more binding effect on the PVP-iodine in the fibres and therefore released less iodine from the samples.
Whatever the reasons and variations observed, the important conclusion was that a process has been established for producing fibres containing available iodine to commercial levels.

Unless defined otherwise all technical and scientific terms used herein have the same meaning as commonly understood to one of ordinary skill in the art to which the presently described subject matter pertains.

Where a range of values is provided, for example, concentration ranges, percentage ranges, or ratio ranges, it is understood that each intervening value, to the tenth of the unit of the lower limit, unless the context clearly dictates otherwise, between the upper and lower limit of that range and any other stated or intervening value in that stated range, is encompassed within the described subject matter. The upper and lower limits of these smaller ranges may independently be included in the smaller ranges, and such embodiments are also encompassed within the described subject matter, subject to any specifically excluded limit in the stated range. Where the stated range includes one or both of the limits, ranges excluding either or both of those included limits are also included in the described subject matter.

1. A swellable fibre formed by a method of extrusion of a primary polymer comprising the steps of:
creating an aqueous dope solution containing a primary polymer and povidone-iodine;
spinning or extruding the aqueous dope solution into a coagulation bath via a spinnerette having a plurality of holes to form an extruded multi filament fibre; and
drawing the fibre from the coagulation bath.

2. The fibre as claimed in claim 1 wherein the primary polymer comprises any one or a combination of:
a polysaccharide;
a polysaccharide based material;
a hydrocolloid forming material; and/or
a polysaccharide/polyvinylpyrrolidone.

3. The fibre as claimed in claim 1 wherein the primary polymer comprises any one or a combination of:
an alginate;
chitosan;
chitin;
pectin;
carboxymethyl cellulose;
hydroxypropyl methylcellulose;
gellan;
konjac; and/or
psyllium.

4. The fibre as claimed in claim 1 wherein the primary polymer is a high M or a high G alginate.

5. The fibre as claimed in claim 1 wherein a concentration of the povidone-iodine in the fibre is in a range of 0.5 to 20 wt % based on a total wt % of the fibre, and optionally wherein a moisture content in the fibre is in the range of 10 to 60 wt % based on a total wt % of the fibre.

6. (canceled)

7. A swellable fibre comprising:
an elongate multi filament body formed from a plurality of intertwined filaments, each filament comprising a primary polymer and povidone-iodine incorporated within the respective filament.

8. The fibre as claimed in claim 7 wherein the primary polymer comprises any one or a combination of:
a polysaccharide;
a polysaccharide based material;
a hydrocolloid forming compound;
a polysaccharide/polyvinylpyrrolidone;
an alginate;
chitosan;
chitin;
pectin;
carboxymethyl cellulose;
hydroxypropyl methylcellulose;
gellan;
konjac; and/or
psyllium.

9. The fibre as claimed in claim 7 wherein the primary polymer comprises a high G-alginate or a high-M alginate and a povidone-iodine concentration in the range of 0.5 to 20_wt % based on a total wt % of the fibre.

10. The fibre as claimed in claim 7 further comprising a water-soluble control compound configured to control a release of iodine from the material, optionally wherein the water-soluble control compound comprises a polyether, an alkyl ether, a compound having a C—O—C linkage, a glycol or glycol combination thereof.

11. (canceled)

12. The fibre as claimed in claim 7 wherein the povidone-iodine is present internally within the multi filament fibre at or towards a core of each filament of the multi filament fibre and at an external surface of the multi filament fibre optionally wherein a moisture content in the fibre is in the range of 10 to 60 wt % based on a total wt % of the fibre.

13. (canceled)

14. A method of forming a swellable polymer based fibre comprising:
creating an aqueous dope solution containing a primary polymer and povidone-iodine;
spinning of extruding the aqueous dope solution into a coagulation bath via a spinneret having a plurality of holes to form an extruded multi filament fibre; and
drawing the fibre from the coagulation bath.

15. The method as claimed in claim 14 wherein the aqueous dope solution further comprises a water-soluble control compound comprising a polyether, an alkyl ether, a compound having a C—O—C linkage a glycol or a combination thereof.

16. The method as claimed in claim 14 wherein the coagulation bath comprises an aqueous solution containing calcium chloride and any one or a combination of polyethylene glycol, propylene glycol or isopropanol, and wherein the coagulation bath optionally further comprises povidone-iodine.

17. (canceled)

18. The method as claimed in claim 14 wherein the primary polymer comprises any one or a combination of:
a polysaccharide;
a polysaccharide based material;
a hydrocolloid forming material; and/or
a polysaccharide/polyvinylpyrrolidone.

19. The method as claimed in claim 14 wherein the primary polymer comprises any one or a combination of:
an alginate;
chitosan;
chitin;
pectin;
carboxymethyl cellulose;
hydroxypropyl methylcellulose;
gellan;
konjac; and/or
psyllium.

20. The method as claimed in claim 14 wherein the primary polymer is high M and/or high G-alginate.

21. The method as claimed in claim 14 wherein the step of drawing the fibre from the coagulation bath comprises passing the fibre into at least one washing bath containing a washing liquid, optionally wherein the washing liquid comprises acetone and/or isopropanol, and optionally wherein the washing liquid further comprises any one or a combination of polyethylene glycol, propylene glycol, a finishing agent, water and/or povidone-iodine.

22. (canceled)

23. (canceled)

24. The method as claimed in claim 21 comprising passing the extruded fibre into a plurality of washing baths in-series, optionally wherein at least some of the washing baths have different washing liquids with different respective constituents.

25. (canceled)

26. (canceled)

27. A material comprising the swellable fibre as claimed in claim 1.

28. The material as claimed in claim 27 further defined as being one of:
a wound dressing material;
a nasal packing material;
a dental packing material;
a suture; or
a seton.
